(19) United States
(12) Patent Application Publication
(10) Pub. No.: US 2010/0028444 A1
(43) Pub. Date: Feb. 4, 2010

(54) USE OF WATER-DISPERSIBLE CAROTENOID NANOPARTICLES AS TASTE MODULATORS, TASTE MODULATORS CONTAINING WATER-DISPERSIBLE CAROTENOID NANOPARTICLES, AND, METHOD FOR TASTE MODULATION

(75) Inventors: Markus Matuschek, Weinheim (DE); Andreas Ernst, Worms (DE); Christian Köpsel, Weinheim (DE); Martin B. Jager, Enkenbach-Alsenborn (DE); Alice Kleber, Bensheim (DE); Michael Krohn, Lorsch (DE); Holger Zinke, Zwingenberg (DE)

Correspondence Address: CONNOLLY BOVE LODGE & HUTZ, LLP, P O BOX 2207, WILMINGTON, DE, 19899, US

(73) Assignee: BASF SE, LUDWIGSHAFEN (DE)

(21) Appl. No.: 12/443,266

(22) PCT Filed: Feb. 25, 2008
(86) PCT No.: PCT/EP08/52273

§ 371 Date: Mar. 27, 2009

(30) Foreign Application Priority Data
Feb. 23, 2007 (EP) 07102977.1
Nov. 26, 2007 (EP) 07121529.7

(51) Int. Cl. A61K 9/14; A23L 1/236; A23L 2/56; A23L 2/60; A23L 1/226; A61K 31/655; A61P 43/00

(52) U.S. Cl. 424/489; 426/534; 426/535; 426/537; 514/150

(57) ABSTRACT

Use of at least one type of water-dispersible carotenoid nanoparticles as taste modulators in compositions of matter; process for taste modulation of compositions of matter in which at least one type of water-dispersible carotenoid nanoparticles is added to compositions of matter; and also taste modulators for compositions of matter comprising

(A) at least one type of water-dispersible carotenoid nanoparticles and
(B) at least one azo compound, comprising at least one azo group.

### FIELD OF THE INVENTION

The present invention relates to the novel use of water-dispersible carotenoid nanoparticles as taste modulators, in particular for reduction of bitter taste and aftertaste in compositions of matter, preferably in foods, drinks, articles consumed for pleasure, sweetening agents, animal feeds and cosmetics, preferably in foods, drinks, articles consumed for pleasure, sweetening agents, animal feeds, cosmetics and pharmaceuticals which comprise at least one HIS (High Intensity Sweetener).

In addition, the present invention relates to a novel process for taste modulation, in particular for reduction of bitter taste and aftertaste of compositions of matter, preferably foods, drinks, articles consumed for pleasure, sweetening agents, animal feeds and cosmetics, preferably foods, drinks, articles consumed for pleasure, sweetening agents, animal feeds, cosmetics and pharmaceuticals which comprise at least one HIS (High Intensity Sweetener) in which at least one type of water-dispersible carotenoid nanoparticles is used as taste modulator.

Not least, the present invention relates to novel taste modulators comprising at least one type of water-dispersible carotenoid nanoparticles.

### PRIOR ART

Compositions of matter such as foods, drinks, articles consumed for pleasure, sweetening agents, animal feeds, cosmetics and pharmaceuticals frequently comprise taste substances which are in principle unwanted or are too dominant or too low in the intensity in which they are present. In the sector of sweeteners, frequently, in addition to the sweet taste impressions, further taste impressions such as, for example, a metallic, chemical, bitter or synthetic taste or aftertaste occurs, which adversely affect the overall taste impression of the composition to be sweetened. In the context of the present invention, taste is taken to mean the immediate taste impression which is formed while the composition is situated in the mouth. Aftertaste is taken to mean the taste perception after swallowing, in particular after a waiting time of about 30 seconds.

For example, caffeine in tea or coffee, and also hop extracts in beer, are natural bitter substances which, however, in too high a concentration cause an adverse taste impression. In special bitter drinks such as, for example, tonic water or bitter lemon, a characteristic bitter taste caused by the additive quinine is desired to a particular extent.

Fruit juices, in particular orange juice, suffer from impairment of the taste by, e.g., flavonoid glycosides, which have a bitter taste.

Sugar-free drinks which are admixed with sweetening agents likewise exhibit adverse taste attributes, inter alia a bitter taste or aftertaste. Mixing of various sweetening agents reduces the adverse taste impression and optimizes the favorable attributes. However, it is not possible to imitate the sugar taste completely. In addition, individual sweetening agents such as aspartame (ASP) are, in certain cases, incompatible or chemically unstable. Because of the favorable properties of ACK, a higher dosage of this sweetening agent is sought. This higher dosage, however, is only possible with restrictions owing to the bitter taste of this sweetening agent in relatively high concentrations. This is because, in particular, the sweeteners saccharin and ACK have bitter taste attributes, especially in high concentrations.

Many pharmaceutical active compounds, in particular ibuprofen, also have a strongly bitter taste which leads to reduction in acceptance when the active compound is taken.

For reduction of the natural bitter taste, for example of tea, coffee or orange juice, these foods and articles consumed for pleasure are either enzymatically treated in order to destroy the bitter tasting substances, or the bitter substance is removed by decaffeination in the case of caffeine in tea and coffee.

A further possibility of modifying the taste impression is addition of taste modulators to the desired foods, drinks, articles consumed for pleasure, animal feeds, sweetening agents, cosmetics and pharmaceuticals.

It is therefore desirable to find substances which suppress or reduce the unpleasant taste impressions, and also can amplify in a targeted manner desired taste impressions.

In particular in the sector of pharmaceutical active compounds, a great number of substances which, in particular, modify bitterness, are known. Thus, for example, the bitter taste of ibuprofen is masked by polylysine and polyarginine (cf. international patent application WO 2003/086293), by meglumine salt (cf. U.S. Pat. No. 5,028,625), by sodium chloride or sodium-saccharin (cf. international patent application WO 2003/0475550) or by hydroxypropyl-beta-cyclodextrin or chewable methacrylic acid copolymers (cf. Modifying Bitterness, Mechanism, Ingredients And Applications, Glenn Roy, 1997) in order to facilitate intake by patients. The bitterness of caffeine may also be reduced by a multiplicity of taste modulators such as, for example, glutamic acid, dicalcium disalicylate, starch, lactose, manitol and also by phosphatidic acid and beta-lactoglobulin (cf. Glenn Roy, 1997) and in addition by hydroxybenzamides, in particular hydroxybenzoic acid vanillylamide (cf. Ley et al., Journal of Agricultural & Food Chemistry, 2006).

Further substances which have been used for reduction of a bitter taste in general and in particular in pharmaceuticals and foods are lecithin, ascorbate and citrate (cf. Japanese patent application JP 2001226293), esters of mono- or diglycerides such as glycerol monostearate and polycarboxylic acids such as succinic acid (cf. European patent application EP 0 732 064 A1), hydroxyflavanones (cf. European patent application EP 1 258 200 A1), 2-phenyl-4-chromanone derivatives (cf. German patent application DE 101 22 898), sodium sulfate hydrate (cf. Japanese patent application JP 02025428). In addition, U.S. Pat. No. 5,637,618 discloses the use of benzoic acid derivatives for reduction of the bitter taste in drinks and also of sweetening agents and of potassium chloride. The bitter taste of potassium chloride is also inhibited using 2,4-dihydroxybenzoic acid, carrageenan and thaumatin (cf. Glenn Roy, 1997; U.S. Pat. No. 5,637,618 and also Japanese patent applications JP 04262758 and JP 07083684).

However, the known taste modulators are not completely satisfactory, in particular when the intention is to use them for reduction of the bitter taste of compositions of matter such as, for example, foods, drinks, articles consumed for pleasure, sweetening agents, animal feeds, cosmetics and pharmaceuticals which comprise at least one HIS, but in particular ACK, but in particular of HIS-comprising soft drinks. In this case their bitterness-reducing activity is frequently insufficient. If, for this reason, the concentration of the known taste modulators is increased in order to achieve sufficient activity, unwanted physical and/or chemical interactions with the remaining components of the respective compositions and/or adverse effects, in particular impairment up to complete distortion of the characteristic taste impression thereof can occur.

Water-dispersible carotenoid nanoparticles, processes for production thereof and use thereof are known per se.

For instance, water-dispersible carotenoid nanoparticles follow, for example, from European patent application EP 0 832 569 A2, the paper by Dieter Horn and Jens Rieger, “Organische Nanopartikel in wässriger Phase—Theorie, Experiment und Anwendung” [Organic nanoparticles in aqueous phase—theory, experiment and application], in Angewandte Chemie, 2001, volume 113, pages 4460 to 4492, or the textbook by J. C. Bauernfeind, “Carotenoids as Colorants and Vitamin A Precursors. Technological and Nutritional Applications”, Chapter 2, J. C. Bauernfeind and H. Kläui, “Carotenoids as Food Color”, pages 92 to 95, Academic Press, ISBN 0-12-082850-2, 1981. Preferably the carotenoid nanoparticles are spherical or spheroidal particles which have a particle size <1 μm, preferably determined on the basis of electron microscope images. The water-dispersible carotenoid nanoparticles are present in formulations or suspensions which additionally comprise additives such as oils, protective colloids, stabilizers or emulsifiers. In this case the carotenoids can be crystalline or amorphous.

These water-dispersible carotenoid nanoparticles and the carotenoid-comprising formulations which comprise them can be used as additives for foods, for example baking mixes or pudding powders, or as dry powders for producing formulations for food supplementation with vitamins in the human and animal sectors and also for producing pharmaceutical formulations. Owing to their good cold water dispersibility, they are suitable, in particular, as food dyes, especially for soft drinks. The use of these water-dispersible carotenoid nanoparticles and the carotenoid-comprising formulations which comprise them, as taste modulators is not described.

Carotenoid nanoparticles, however, can also be present in carotenoid-comprising formulations which are O/W microemulsions (oil-in-water microemulsions; cf. Römpp Online 2007, “Mikroemulsionen” [Microemulsions]). These O/W microemulsions comprise oil droplets of a diameter <1 μm, where the carotenoids are dissolved in molecularly disperse form. The use of these water-dispersible carotenoid nanoparticles or the O/W microemulsions which comprise them as taste modulators is not known.

However, carotenoid nanoparticles can also be produced by milling aqueous suspensions comprising carotenoids, modified starch and a sugar such as sucrose, and subsequent drying, as is described, for example, in German patent application DE 10 2005 030 952 A1. These carotenoid nanoparticles are suitable as additives to food preparations, for example for coloring foods such as drinks, as means for producing pharmaceutical and cosmetics preparations, and also for producing food supplement formulations, for example multivitamin formulations in the human and animal sectors. The use as taste modulators is not described.

However, carotenoid nanoparticles can also be used as aqueous solubilisates in mixed micelles of a micelle size <100 nm (cf. Römpp Online 2007, “Solubilisation” and “Micellen” [Micelles]). Examples of such aqueous solubilisates are disclosed by European patent applications EP 0 800 825 A1 and EP 0 848 913 A2. These carotenoid nanoparticles or their aqueous solubilisates are used for injection purposes for parenteral administration and for coloring foods and pharmaceuticals, in particular for coloring drinks which must remain visually clear. The use as taste modulators is not described.

The use of beta-carotene as dye in low-calorie soft drinks which comprise sweetening agents such as ASP and ACK is known. One example of such soft drinks is Coca-Cola light Sango® having the taste of blood oranges. It is not known in what form beta-carotene is added to the soft drink.

The joint use of azo dyes such as Yellow 6 and Red 40 for coloring soft drinks which comprise ACK as HIS is likewise known. One example of such a product is Diet Sunkist® Orange Soda. It is not known whether the azo dyes used also cause a reduction of the bitter taste and bitter aftertaste of ACK. It is still less known whether, by means of the joint use of azo dyes and carotenoids, a taste-modulating action which is possibly present may be amplified.

In addition, the use of azo dyes such as E 110 and E 129 together with beta-carotene in the food such as bakery products and confectionery and also in instant drink powders is also known, however always using a combination of sweetening agents such as ACK and ASP with sugar in the case of confectionery and instant drink powders and starches in the case of bakery products.

Therefore, the abovedescribed prior art including the products available on the market give no cause or indications as to how the abovedescribed problems could be solved.

### OBJECT OF THE INVENTION

Accordingly, the object of the present invention was to provide substances which may be used outstandingly as taste modulators, in particular for reduction of bitter taste and bitter aftertaste in compositions of matter, preferably in foods, drinks, articles consumed for pleasure, sweetening agents, animal feeds, cosmetics and pharmaceuticals, preferably in foods, drinks, articles consumed for pleasure, sweetening agents, animal feeds, cosmetics and pharmaceuticals which comprise at least one HIS (High Intensity Sweetener), in particular ACK.

In this case, these substances, in their novel use as taste modulators, must cause no unwanted physical and/or chemical interactions with the remaining components of the respective compositions of matter, in particular the foods, drinks, articles consumed for pleasure, sweetening agents, animal feeds, cosmetics and pharmaceuticals. In addition, they must not adversely affect the characteristic taste impression thereof, in particular they must not impair it or completely distort it.

In addition, these substances for the novel use as taste modulators must be producible on the basis of substances which are known per se, readily obtainable and inexpensive.

Furthermore, the object of the present invention was to find a novel process for taste modulation, in particular for reduction of bitter taste and bitter aftertaste of compositions of matter, preferably for taste modulation of foods, drinks, articles consumed for pleasure, sweetening agents, animal feeds, cosmetics and pharmaceuticals, preferably foods, drinks, articles consumed for pleasure, sweetening agents, animal feeds, cosmetics and pharmaceuticals which comprise at least one HIS (High Intensity Sweetener), in particular ACK.

The novel process for taste modulation must have the effect that the taste modulators used do not cause any unwanted physical and/or chemical interactions with the remaining components of the respective compositions, in particular the foods, drinks, articles consumed for pleasure, sweetening agents, animal feeds, cosmetics and pharmaceuticals, and the characteristic taste impression is not adversely affected, in particular is not impaired or completely distorted.

### SOLUTION ACCORDING TO THE INVENTION

Accordingly, the novel use of water-dispersible carotenoid nanoparticles as taste modulators in compositions of matter has been found.

Hereinafter this novel use of water-dispersible carotenoid nanoparticles is termed “use according to the invention”.

In addition, the novel process for taste modulation of compositions of matter has been found in which at least one type of water-dispersible carotenoid nanoparticles is added to the compositions of matter.

Hereinafter, the novel process for taste modulation of compositions of matter is termed “process according to the invention”.

Not least, the novel taste modulators have been found for compositions of matter which

(A) comprise at least one type of water-dispersible carotenoid nanoparticles and
(B) at least one azo compound comprising at least one azo group.

Hereinafter the novel taste modulators for compositions of matter are termed “taste modulators according to the invention”.

### ADVANTAGES OF THE INVENTION

In relation to the prior art, it was surprising, and not predictable by a person skilled in the art, that the object of the present invention could be solved by means of the use according to the invention, the process according to the invention and the taste modulators according to the invention.

It was especially surprising that the water-dispersible carotenoid nanoparticles to be used according to the invention, but in particular the taste modulators according to the invention, could be used outstandingly as taste modulators, in particular for reduction of bitter taste and bitter aftertaste in compositions of matter, preferably in foods, drinks, articles consumed for pleasure, sweetening agents, animal feeds agents, cosmetics and pharmaceuticals, preferably in foods, drinks, articles consumed for pleasure, sweetening agents, animal feeds, cosmetics and pharmaceuticals which comprise at least one HIS (High Intensity Sweetener), in particular ACK.

The water-dispersible carotenoid nanoparticles to be used according to the invention, but in particular the taste modulators according to the invention, showed no unwanted physical and/or chemical interactions with the remaining components of the respective compositions of matter, in particular the food, drinks, articles consumed for pleasure, sweetening agents, animal feeds, cosmetics and pharmaceuticals. In addition, they did not adversely affect the characteristic taste impression thereof, in particular did not impair or completely distort it.

In addition, the water-dispersible carotenoid nanoparticles to be used according to the invention, but in particular the taste modulators according to the invention, could be produced in a simple manner based on readily available and inexpensive substances which are known per se.

In addition, the process according to the invention had the effect that the taste modulators used did not cause any unwanted physical and/or chemical interactions with the remaining components of the respective compositions, in particular the foods, drinks, articles consumed for pleasure, sweetening agents, animal feeds, cosmetics and pharmaceuticals, and the characteristic taste impression was not adversely affected, in particular was not impaired or even completely distorted.

It was especially surprising that the taste modulation of a given composition of matter by the use according to the invention, the process according to the invention and the taste modulators according to the invention was outstandingly reproducible, which, precisely in regard to the production of mass products such as foods, drinks, articles consumed for pleasure, sweetening agents, animal feeds, cosmetics and pharmaceuticals, is a very particular advantage.

### DETAILED DESCRIPTION OF THE INVENTION

For the use according to the invention, the process according to the invention and the taste modulators according to the invention, the water-dispersible carotenoid nanoparticles to be used according to the invention are critical.

The carotenoid nanoparticles can have any three-dimensional shapes, such as, for example, pyramidal, cubic, octahedral, icosahedral, platelet-like, needle-shaped, cylindrical, spherical or spheroidal shapes. Preferably, they have a spherical or spheroidal shape. More preferably, the spherical shape is in the form of a ball.

The particle size of the carotenoid nanoparticles is less than 1 μm, preferably between 10 and 900 nm, more preferably between 20 and 700 nm, particularly preferably between 20 to 600 nm, very particularly preferably between 20 to 500 nm, and in particular between 20 and 300 nm. Preferably, the particle size is determined from electron microscope images.

In this case the spheroidal particles are preferably longitudinally prolate and more preferably here have a length of 200 to 300 nm and a thickness 100 to 150 nm. Preferably the median particle size of the carotenoid nanoparticles, determined by quasielastic light scattering, is between 10 and 900 nm, preferably 20 to 700 nm, particularly preferably 20 to 500 nm, in particular 20 to 300 nm.

The essential component of the carotenoid nanoparticles is at least one, in particular one, carotenoid. “Carotenoids” is the collective name for carotenes, a group of highly unsaturated, aliphatic and alicyclic hydrocarbons and their manifold modified derivatives. The majority are tetraterpenes which are made up of 8 isoprene units. The color of the carotenoids (yellow to red) is based on their polyene structure having numerous conjugated double bonds. From the basic backbone having 40 carbon atoms are derived not only the xanthophylls substituted by hydroxyl or oxo groups, but also apo-, nor- or seco-carotenoids which have shortened chains or open rings, and retrocarotenoids, wherein the double bonds are shifted. However, the carotenoids can also possess carboxyl groups.

Examples of suitable carotenoids are alpha-, beta- and gamma-carotene, lycopene, beta-apo-4′-carotenal, beta-apo-8′-carotenal, beta-apo-12′-carotenal, beta-apo-8′-carotenic acid, zeaxanthin, astaxanthin, violaxanthin, canthaxanthin, citranaxanthin, cryptoxanthin, flavoxanthin, rhodoxanthin, rubixanthin, flucoxanthin, mutatoxanthin, lutoxanthin, auroxanthin, capsanthin, lutein, crocetin, neurosporene, echinenone, adonirubin, torulene, torularhodin, bixin, peridinin and peridinol, wherein the carotenoids comprising hydroxyl groups and carboxyl groups can be esterified. In particular, use is made of beta-carotene (provitamin A).

The carotenoids can be present in the carotenoid nanoparticles in different states of matter.

For instance, the carotenoid nanoparticle can be solid. In this case the carotenoids in the carotenoid nanoparticles can be present in crystalline form and/or X-ray amorphous form, preferably X-ray amorphous form. “X-ray amorphous” means that the crystalline fraction is below 10%. Preferably, the carotenoids in this case have a high fraction of all-trans configuration which is preferably at least 50%, and in particular at least 60%. If the solid carotenoid nanoparticles are used in combination with at least one additive, they are preferably embedded in a matrix of this additive.

In addition, the carotenoids in the carotenoid nanoparticles can also be present in the liquid state. This is the case, in particular, when the carotenoids are dissolved in a liquid, preferably nonpolar, medium such as, for example, an oil, or in the form of solubilized mixed micelles.

The carotenoid nanoparticles can be produced by means of various processes.

Preferably these processes result in liquid or solid formulations comprising carotenoid nanoparticles. Preferably, these are the solid formulations (A1) and (A3), in particular (A1), and the liquid formulations (A2) and (A4), in particular (A2).

The solid formulations (A1) can be produced, for example, using the conventional and known precipitation methods, as are described in European patent application EP 0 832 569 A2, the paper by Dieter Horn and Jens Rieger, “Organische Nanopartikel in wässriger Phase—Theorie, Experiment und Anwendung” [Organic nanoparticles in aqueous phase—theory, experiment and application] in Angewandte Chemie, 2001, volume 113, pages 4460-4492, or the textbook by J. C. Bauernfeind, “Carotenoids as Colorants and Vitamin A Precursors. Technological and Nutritional Applications”, Chapter 2, J. C. Bauernfeind and H. Kläui, “Carotenoids as Food Color”, pages 92 to 95, Academic Press, ISBN 0-12-082850-2, 1981. The resultant suspensions of nanoparticles are dried using suitable processes, for example by spray drying, and used in the form of powders (A1). Preferably, the solid formulations (A1) comprise at least one additive. Preferably, in the solid formulations (A1), the carotenoid nanoparticles are embedded in a matrix of at least one additive. Preferably, the carotenoid is present in amorphous form in the carotenoid nanoparticles of interest.

The liquid formulations (A2) are O/W microemulsions, i.e. oil-in-water microemulsions (cf. also Römpp Online 2007, “Mikroemulsionen” [Microemulsions]). The particle size of the disperse phase and the diameter of the oil droplets is <1 μm. Preferably, the diameter of the oil droplets is 10 to 900 nm, more preferably 20 to 700 nm, and in particular 20 to 500 nm.

In the liquid formulations (A2) the carotenoids are dissolved in molecularly disperse form in the oil droplets.

Preferably, the liquid formulations (A2) further comprise at least one additive which stabilizes the O/W microemulsions, in particular at least one polyol.

The solid formulations (A3) can be produced, for example, by milling carotenoid particles in an aqueous suspension and subsequently drying. A suitable process is described, for example, in German patent application DE 10 2005 030 952 A1, page 4, paragraph [0032], to page 5, paragraph [0043].

Preferably, the solid formulations (A3) comprise at least one additive. Preferably, in the formulations (A3), the carotenoid nanoparticles are embedded in a matrix of at least one additive. Preferably, the carotenoid in the carotenoid nanoparticles in question is crystalline.

The liquid formulations (A4) are aqueous solubilisates, wherein the carotenoids are present in mixed micelles of a micelle size <100 nm (cf. Römpp Online 2007, “Solubilisation” and “Micellen” [Micelles]).

Preferably, the liquid formulations (A4) comprise at least one additive, in particular at least one additive which stabilizes the mixed micelles, in particular at least one emulsifier.

Examples of suitable liquid formulations (A4) and processes for their production are disclosed by European patent applications EP 0 800 825 A1, page 2, line 52, to page 3, line 36, and EP 0 848 913 A2, page 2, line 42, to page 3, line 58.

Preferably, the at least one additive which is used for production of the liquid or solid formulations (A1) to (A4), in particular the solid formulations (A1) and the liquid formulations (A2), is an additive which is permitted under food law and/or drugs law. Preferably, the additive is selected from the group consisting of protective colloids, stabilizers against oxidative breakdown, emulsifiers, oils, plasticizers and compositions against caking.

Examples of suitable protective colloids are gelatin, fish gelatin, starch, chemically or enzymatically modified starch, dextrins, plant proteins, pectins, gum Arabic, casein, caseinate, polyvinyl alcohol, polyvinylpyrrolidone, methylcellulose, carboxymethylcellulose and alginates (cf. R. A. Morton, Fat Soluble Vitamins, International Encyclopedia of Food and Nutrition, volume 9, Pergamon Press, 1970, pages 128 to 131). Preferably, they are present in the formulations in an amount of, based on the formulations, 10 to 80% by weight.

Examples of suitable stabilizers are alpha-tocopherol, tertiary butylated hydroxytoluene, tertiary butylated hydroxylanisole, ascorbic acid or ethoxyquin (6-ethoxy-1,2-dihydroxy-2,2,4-trimethylquinoline).

Examples of suitable emulsifiers are ascorbyl palmitate, polyglycerol esters of fatty acids, sorbitan esters of fatty acids, polypropylene glycol esters of fatty acids and lecithin. Preferably, they are used in an amount of up to 200% by weight, preferably 10 to 150% by weight, and in particular 20 to 80% by weight, in each case based on the carotenoids.

Examples of suitable oils are sesame oil, corn germ oil, cottonseed oil, soybean oil, peanut oil and also the esters of medium chain plant fatty acids. Preferably, they are used in an amount of up to 500% by weight, preferably 10 to 300% by weight, and in particular 20 to 100% by weight, in each case based on the carotenoids.

One example of a suitable polyol is glycerol.

Examples of suitable plasticizers are sugar and sugar alcohols such as sucrose, glucose, lactose, invert sugar, sorbitol, mannitol and glycerol. Preferably, they are present in the carotenoid nanoparticles in an amount of 20 to 70% by weight, based on the formulations.

One example of a suitable anticaking agent is tricalcium phosphate.

The carotenoid content of the formulations, in particular the abovementioned liquid or solid formulations (A1) to (A4), can vary widely and can therefore be very well matched to the requirements of the individual case. Preferably, the formulations, based on their total amount, comprise 0.5 to 30% by weight, preferably 1 to 20% by weight, and in particular 5 to 15% by weight, of carotenoids.

In the use according to the invention thereof, the abovementioned carotenoid nanoparticles, preferably the carotenoid nanoparticles present in the above described formulations (A1) to (A4), in particular (A1) and (A2), serve for taste modulation of compositions of matter. In particular they serve for reducing the bitter taste and bitter aftertaste of compositions of matter.

The amount of the carotenoid nanoparticles or formulations thereof, preferably the amount of their formulations (A1) to (A4), in particular the amount of their formulations (A1) and (A2), can in this case vary widely and thus very well adapted to the requirements of the individual case.

Preferably, they are used in an amount such that in solid compositions of matter a concentration of carotenoids of 0.1 to 100 ppm, preferably 1 to 50 ppm, and in particular 2 to 30 ppm, prevails, in each case based on the total amount of a composition of matter.

If the compositions of matter are liquids, they are used in an amount such that, in the liquid compositions of matter, a concentration of carotenoids of 0.1 to 100 mg/l, preferably 1 to 50 mg/l, and in particular 2 to 30 mg/l, prevails.

Preferably, the compositions of matter are foods, drinks, articles consumed for pleasure, sweetening agents, animal feeds and cosmetics, preferably in foods, drinks, articles consumed for pleasure, sweetening agents, animal feeds, cosmetics and pharmaceuticals. The drinks are preferably soft drinks, preferably caffeine-comprising soft drinks, in particular cola drinks.

The compositions of matter preferably comprise at least one High Intensity Sweetener HIS as sweetening agent or sweetener. HIS is taken to mean compounds of synthetic or natural origin which have no physiological calorific value, or negligible calorific value in relation to the sweetening power (non-nutritive sweeteners) and have a sweetening power many times higher than sucrose. The sweetening power of a compound is given by the dilution at which it tastes just as sweet as a sucrose solution (isosweet solution; 0.1 M=4%), i.e. a solution of a sweetener which is diluted 500 times has an isosweet taste like a sucrose solution when the sweetener has a sweetening power of 500.

Examples of suitable HISs are known from Römpp Online 2007, “Süβstoffe” [Sweeteners]. Preferably, the HIS are selected from the group consisting of acesulfame-potassium ACK, aspartame ASP, saccharin and salts thereof, cyclamate and salts thereof, aspartame-acesulfame salt, sucralose, thaumatin, stevia, stevioside and neohesperidin dihydrochalcone, preferably ACK, ASP, saccharin and sucralose, particularly preferably ACK and ASP, in particular ACK. Particularly preferably, ACK is used in drinks.

Preferably, the composition of matter is a low-sugar composition which comprises less than 10 g, preferably less 1 g, of sugar per liter or per kg of composition, in particular a sugar-free composition. Sugars are taken to mean in the present case, in particular, but not exclusively, mono- and disaccharides.

Preferably, the composition of matter is a low-carbohydrate composition which comprises less than 1 g of carbohydrates per liter or per kg of composition, in particular a carbohydrate-free composition.

Advantageously, the composition of matter is a composition having less than 100 kJ, preferably less than 10 kJ, per liter or per kg of composition. Preferably, the composition of matter is a low-fat composition which comprises less than 1 g of fat per liter or per kg of composition, in particular a fat-free composition.

The low-sugar, low-carbohydrate and/or low-fat, in particular sugar-free compositions, are preferably an ACK-sweetened composition, in particular an ACK-sweetened drink.

In the context of the use according to the invention and the process according to the invention, at least one azo compound which reduces the bitter taste and aftertaste and has at least one azo group can be further added to the compositions of matter, in addition to the carotenoid nanoparticles. Preferably, at least two, in particular two, azo compounds are used.

The mixture of at least one type of carotenoid nanoparticles and at least one azo compound is a taste modulator according to the invention. This can be added to the compositions of matter as a finished mixture in the form of an aqueous dispersion or a powder. Or, the individual components of the taste modulator according to the invention are added to the compositions of matter simultaneously or successively.

The amount of the azo compounds can be varied widely and thus very well adapted to the requirements of the individual case. Preferably, they are used in an amount of 0.1 to 100 ppm, more preferably 0.5 to 50 ppm, and in particular 1 to 20 ppm, in each case based on the total amount of a composition of matter. If the composition of matter is a liquid, the carotenoid nanoparticles are preferably used in a concentration of 0.1 to 100 mg/l, more preferably 1 to 50 mg/l, and in particular 2 to 30 mg/l.

In this case the weight ratio of carotenoid nanoparticles to azo compounds can likewise be varied widely and very well matched to the requirements of the individual case. Preferably, the weight ratio of carotenoid nanoparticles to azo compounds is 10:1 to 1:20, more preferably 5:1 to 1:10, and in particular 4:1 to 1:4.

If, which is particularly advantageous according to the invention, two azo compounds are used, their weight ratio can be varied widely and very well matched to the requirements of the individual case. Preferably, the weight ratio is 10:1 to 1:10, more preferably 5:1 to 1:5, and in particular 2:1 to 1:2.

Preferably, the azo groups of the azo compounds are linked to aryl groups and/or aryl groups having heteroatoms, more preferably aryl groups, in particular phenyl groups and/or naphthyl groups. In this case, one or more azo groups can be present in one azo compound. These azo groups can be linked independently of one another to aryl groups and/or aryl groups having heteroatoms, preferably aryl groups, in particular phenyl groups and naphthyl groups.

Preferably, at least one aryl group is at least monosubstituted. In this case one aryl group of an azo group can be unsubstituted while the other is polysubstituted.

Examples of suitable substituents are sulfonic acid groups, nitro groups, alkyl groups, carboxyl groups, hydroxyl groups, ester groups, ether groups, primary and secondary amino groups, amide groups, nitrile groups and halogen atoms, preferably sulfonic acid groups, hydroxyl groups and nitro groups, in particular sulfonic acid groups and hydroxyl groups.

Preferably, the azo compounds are selected from the group consisting of the compounds 1 to 112 listed hereinafter. The azo compound can be ionic or nonionic and can be present in charged or uncharged form.

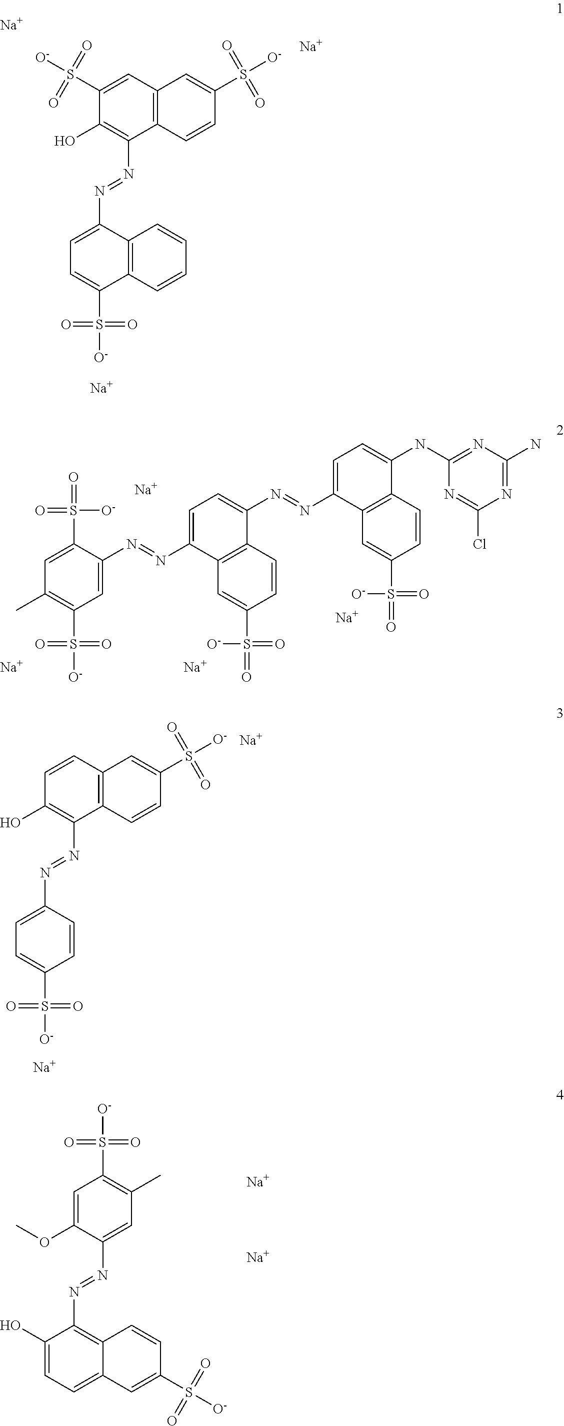

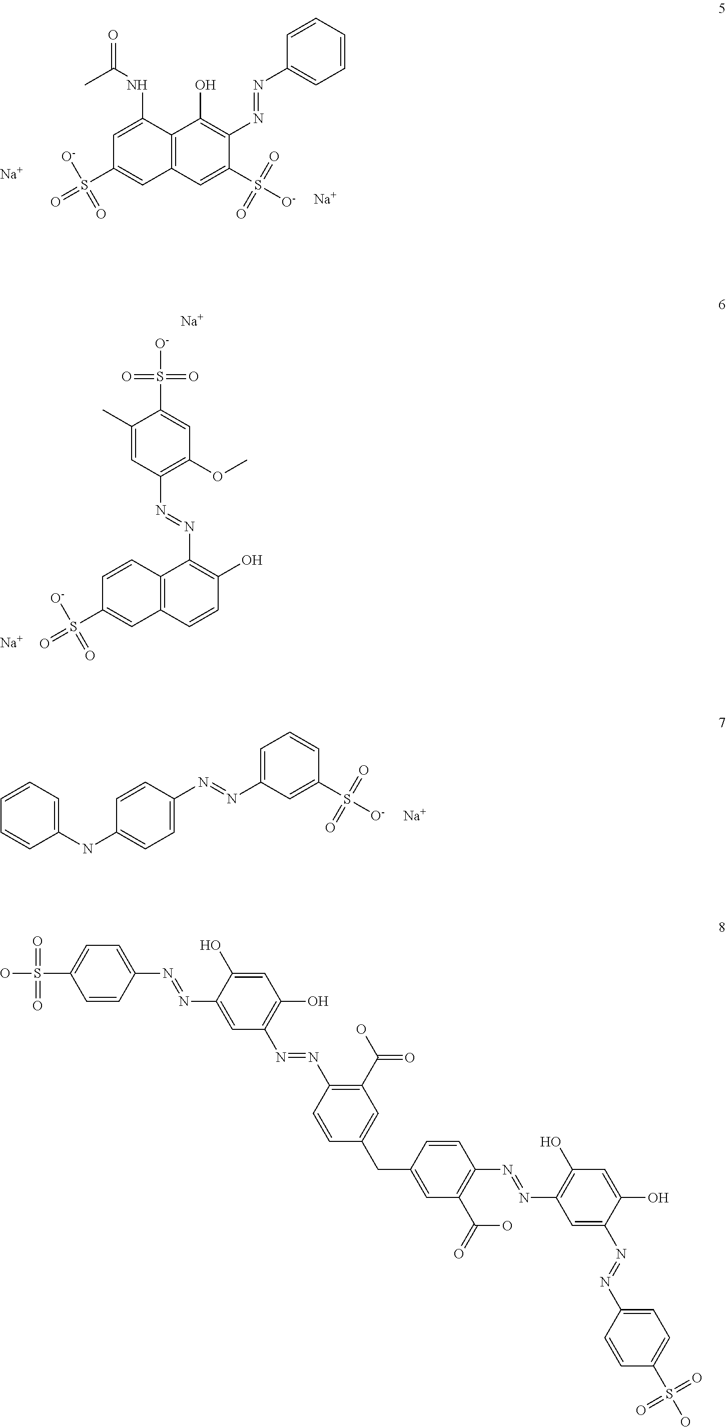

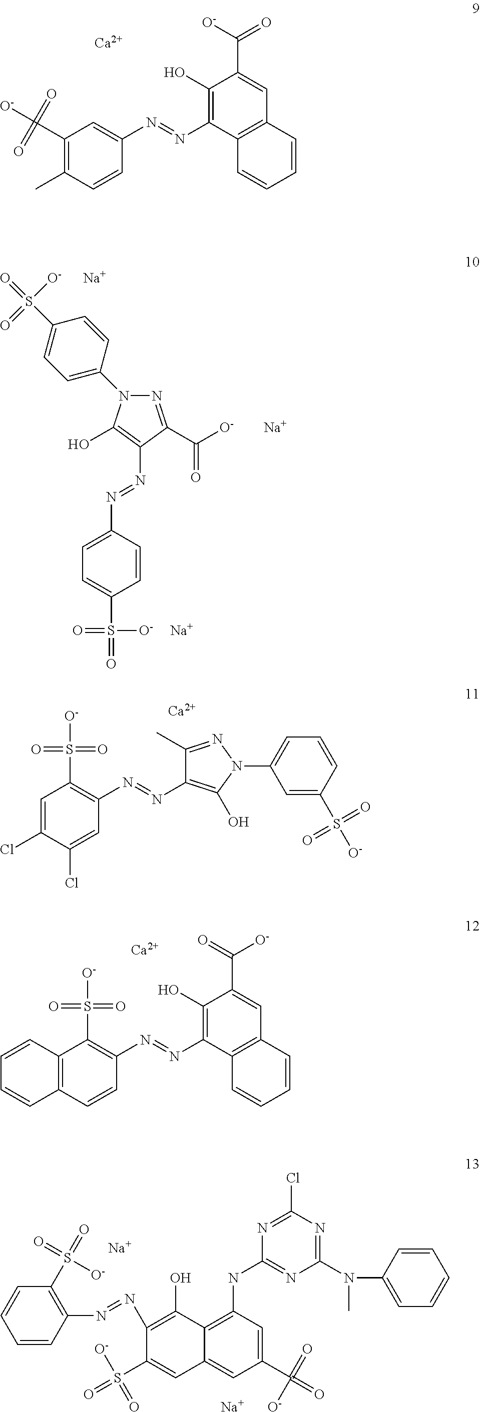

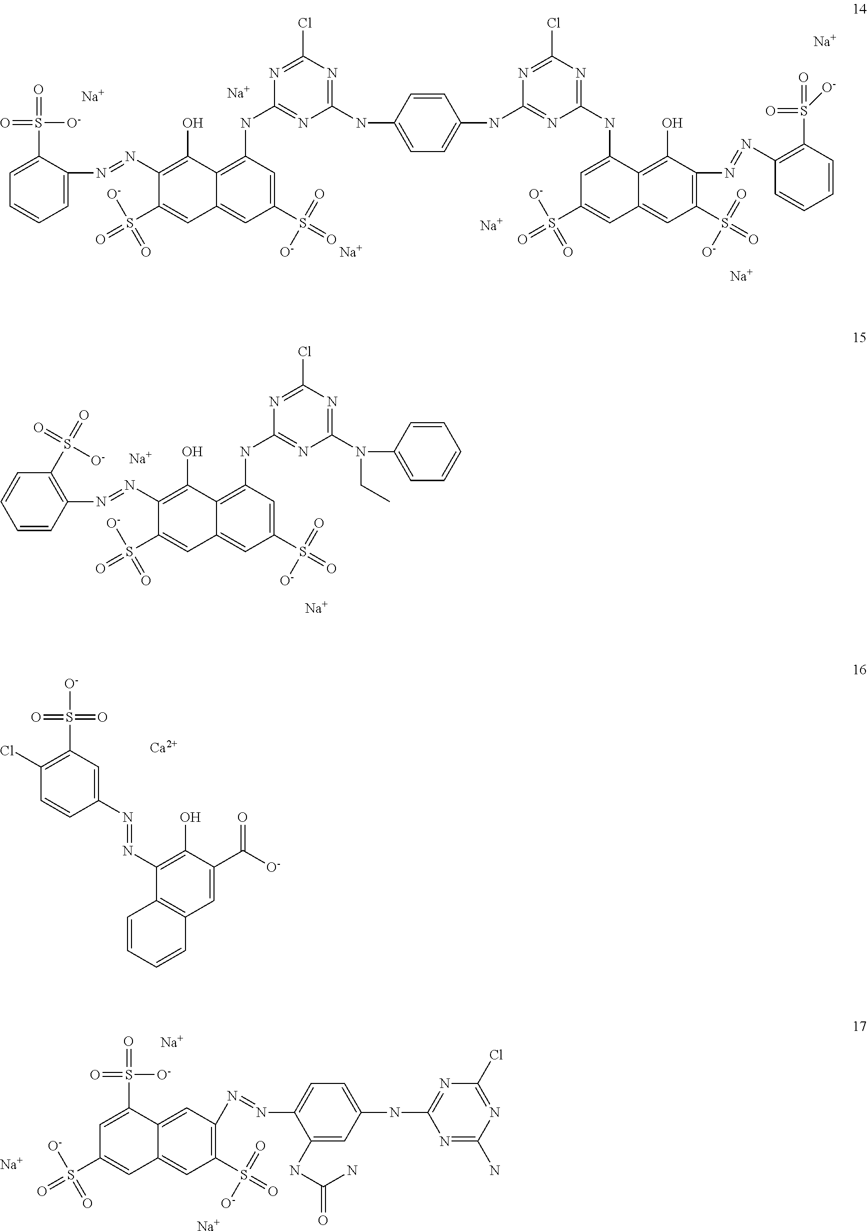

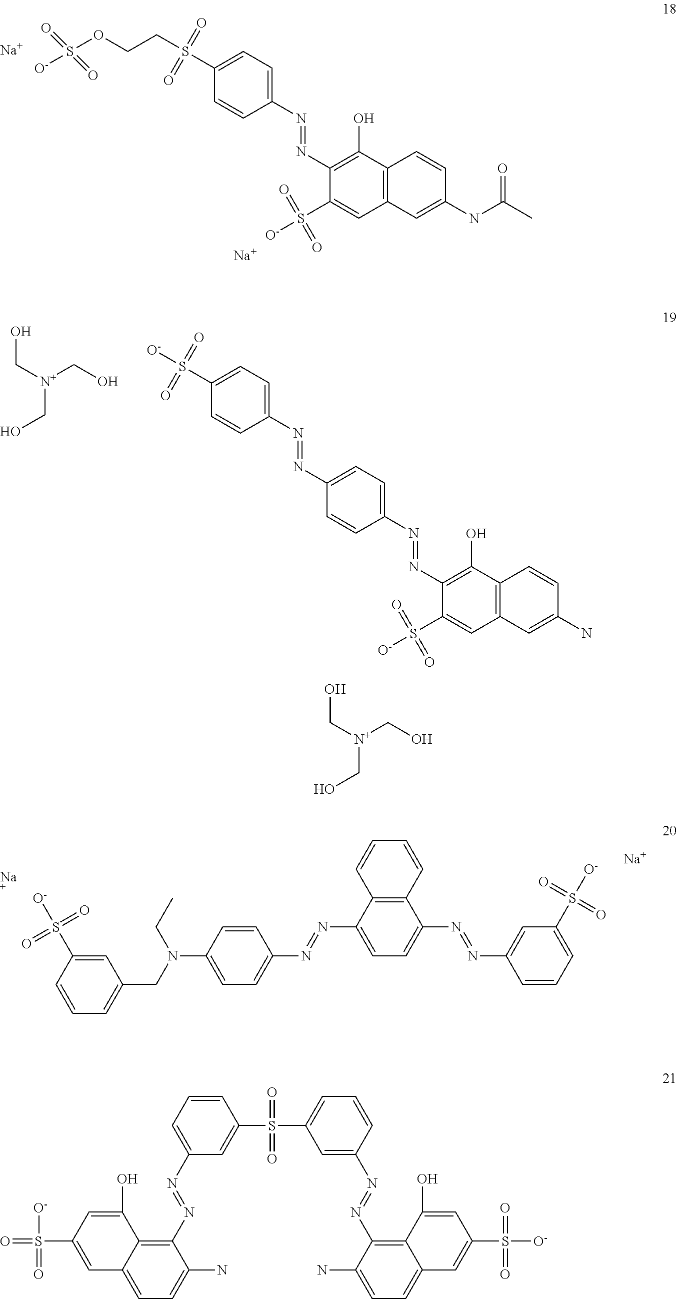

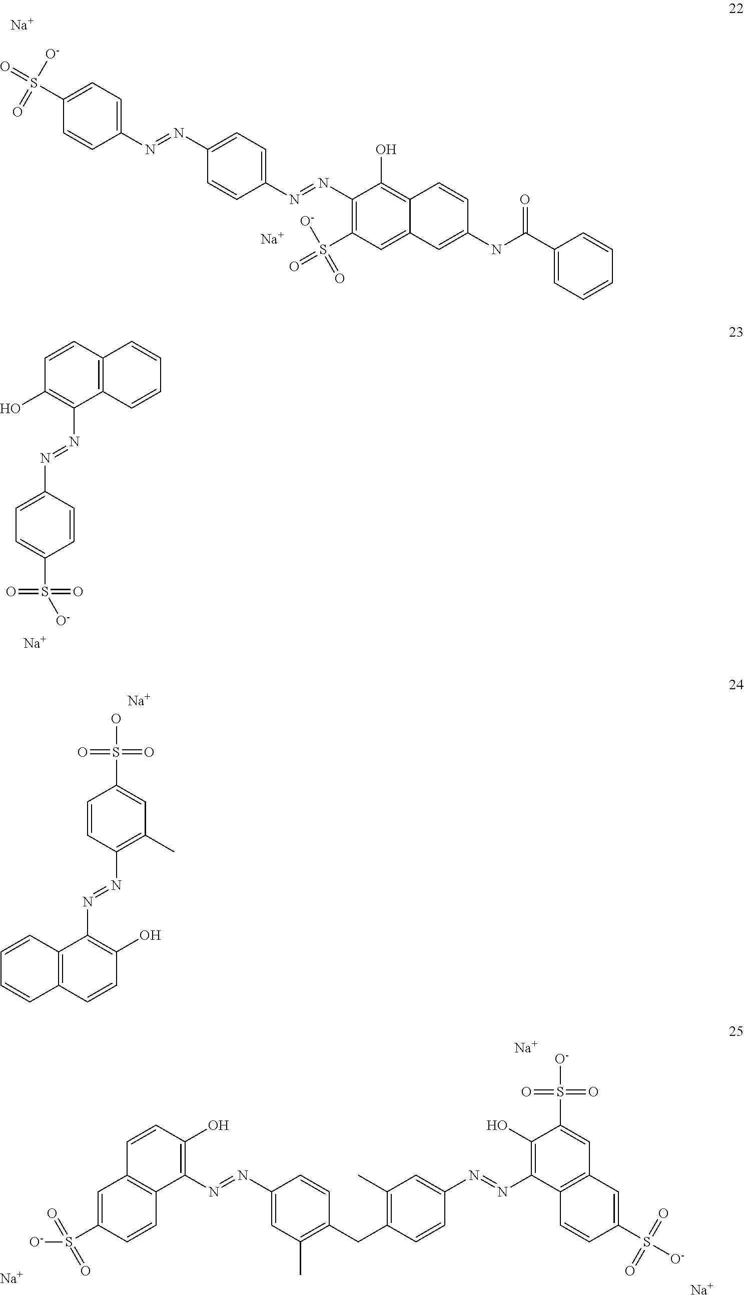

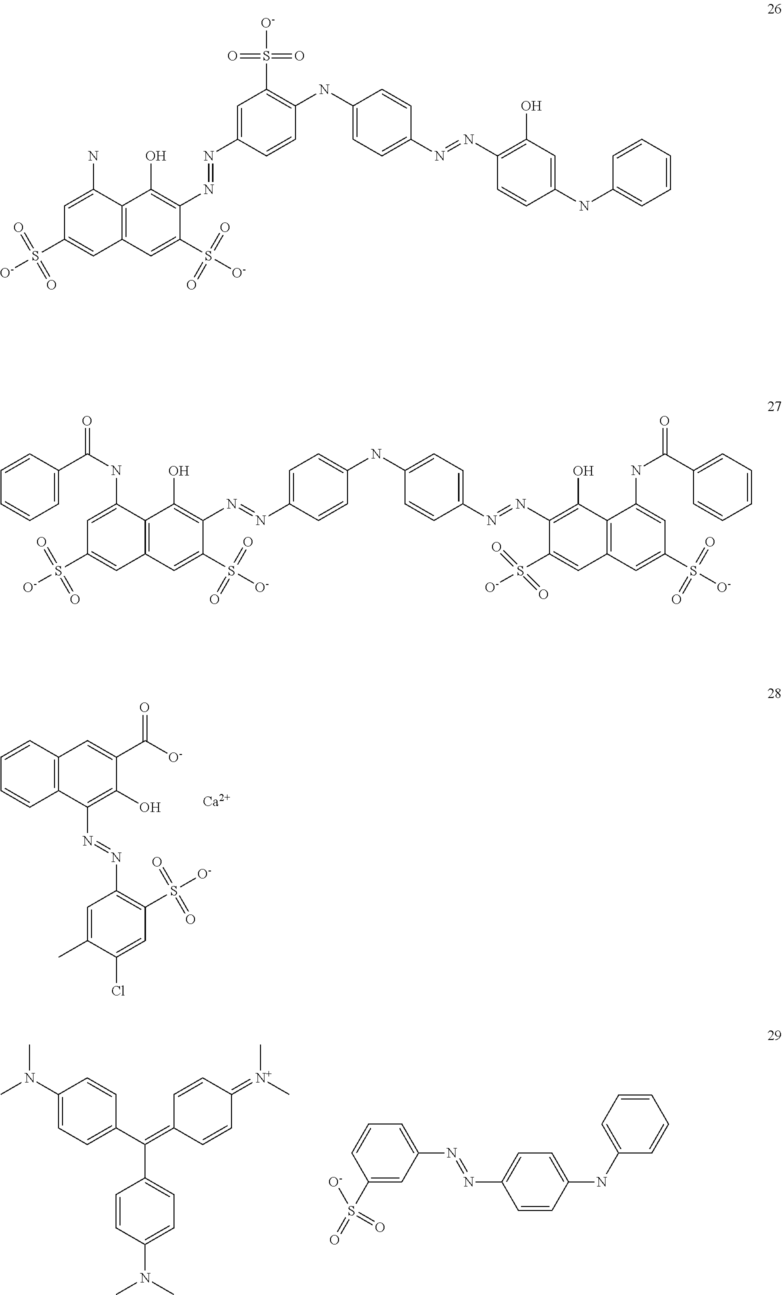

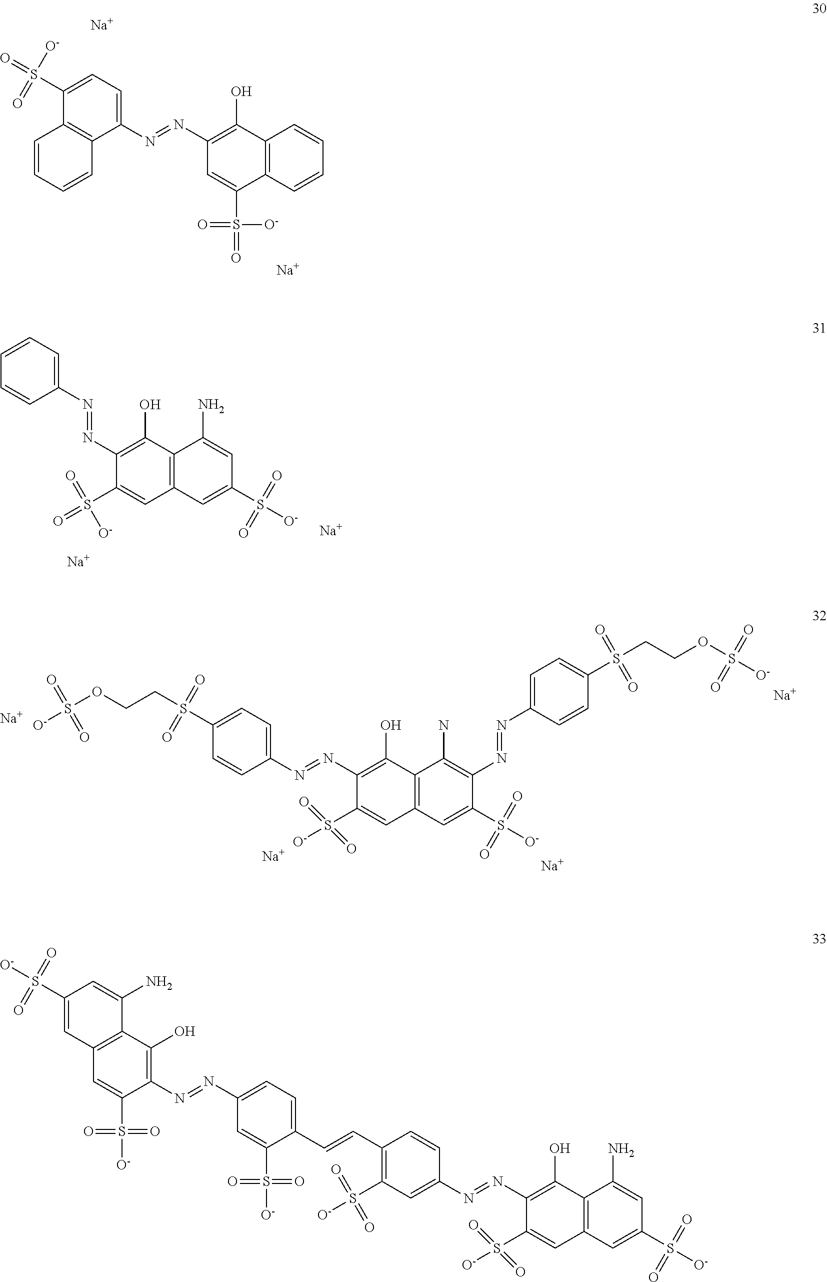

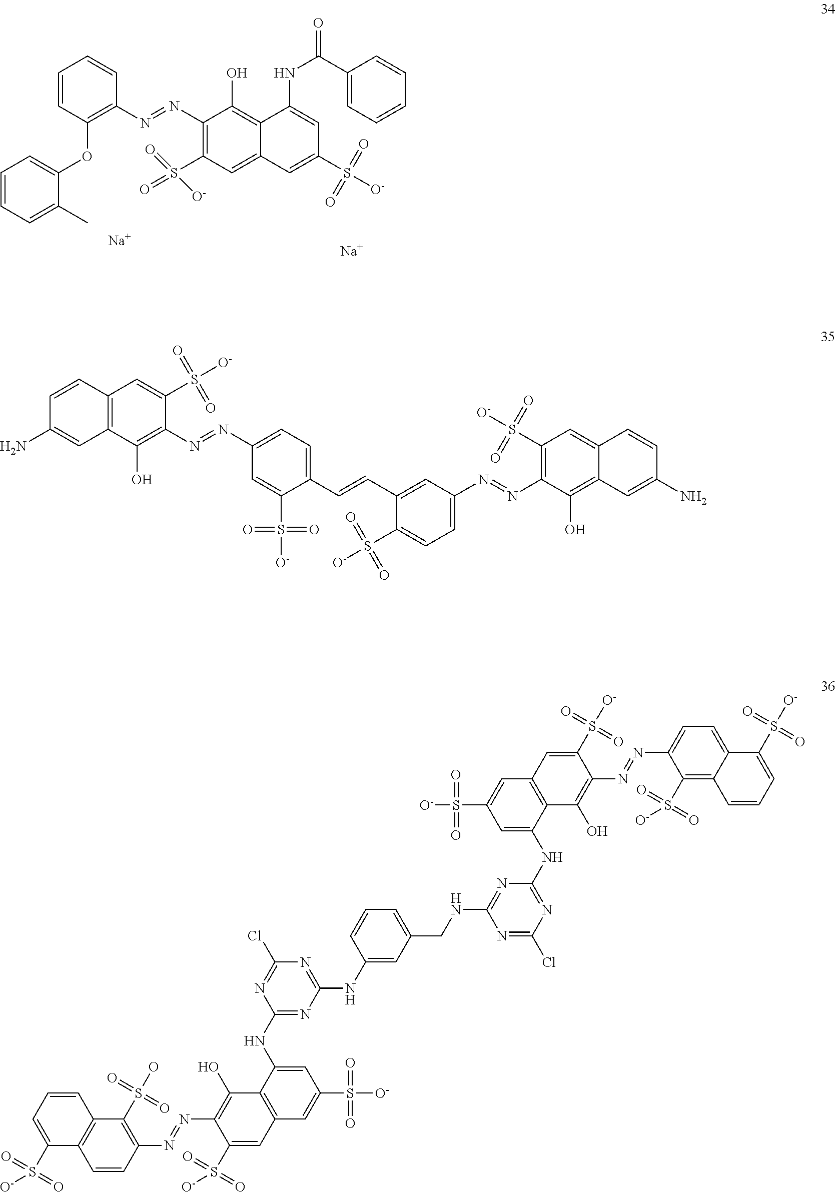

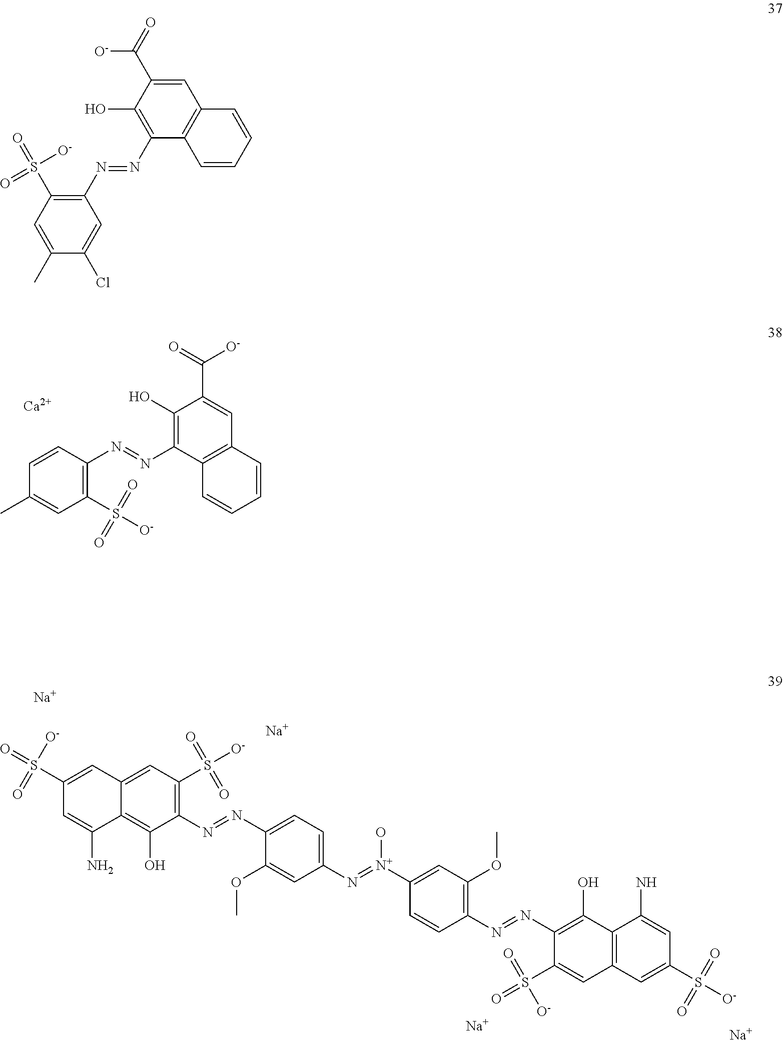

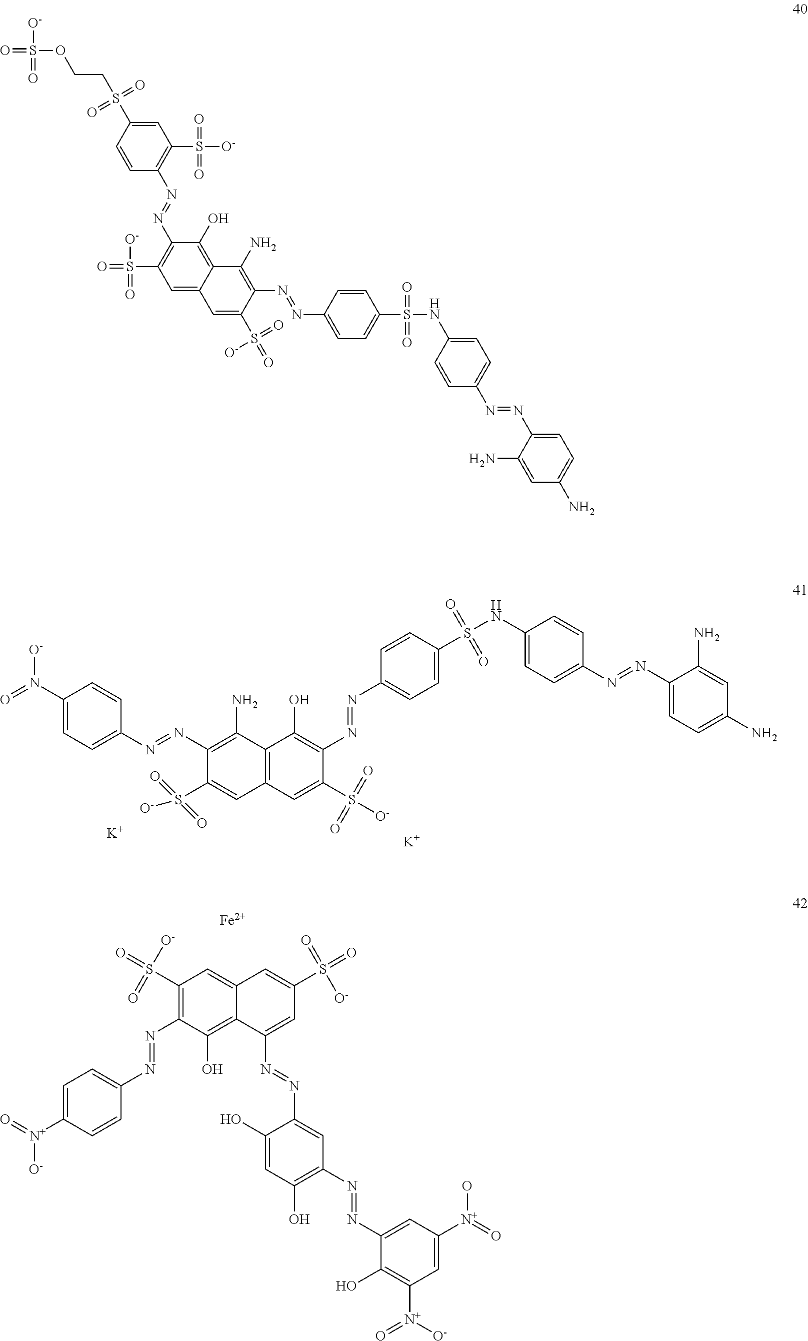

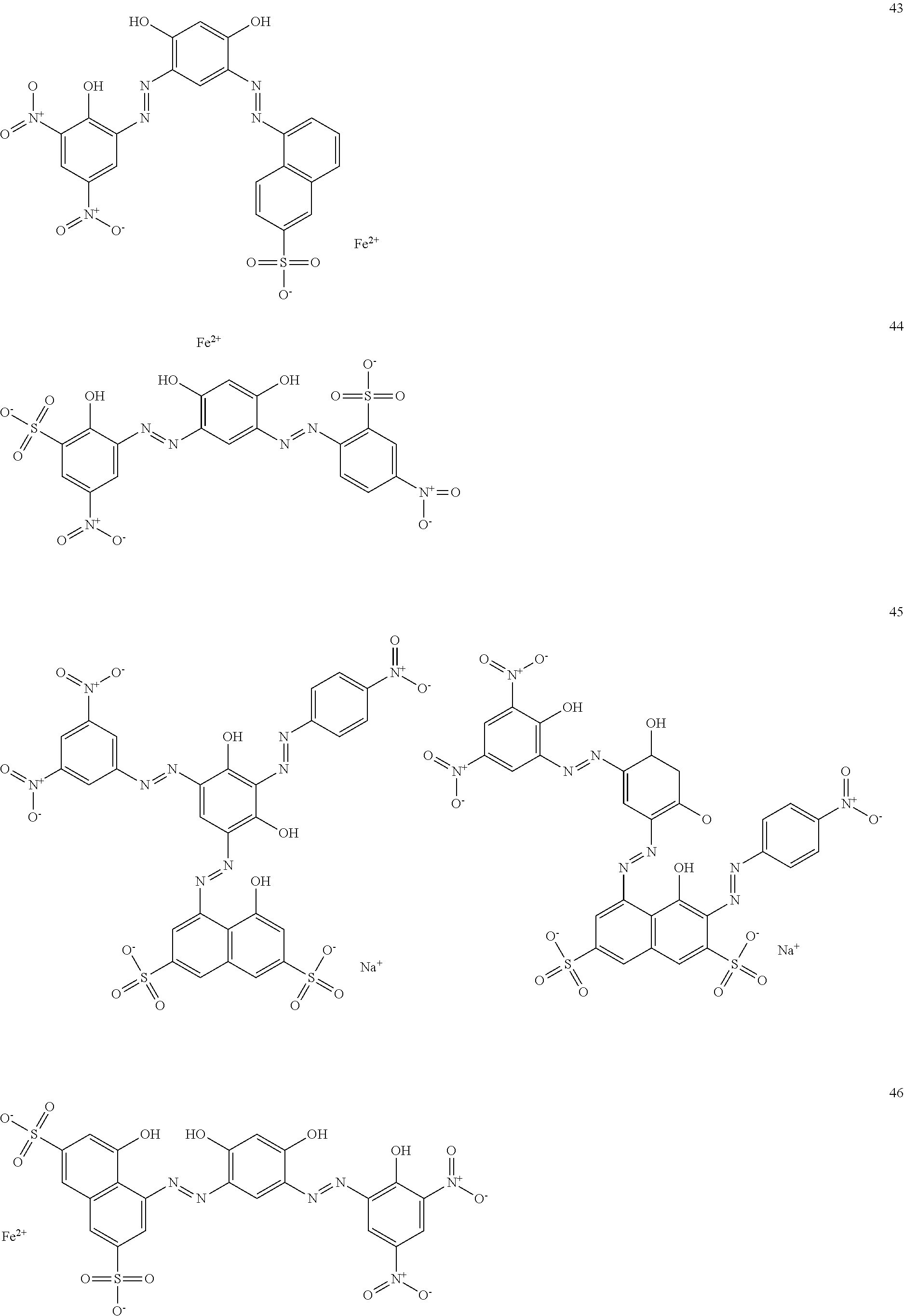

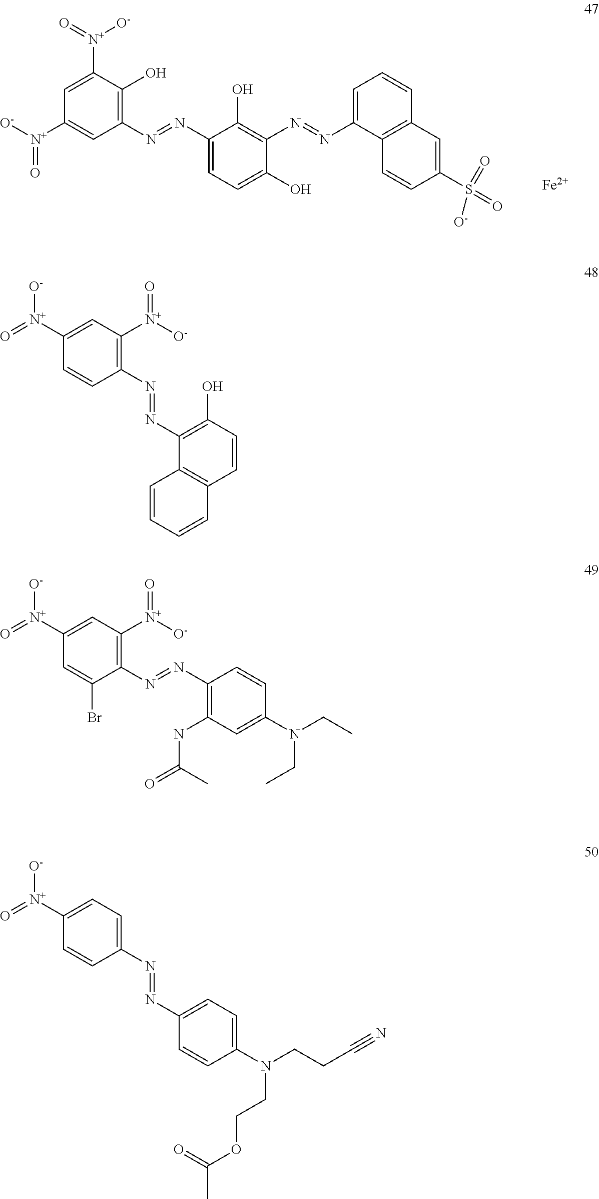

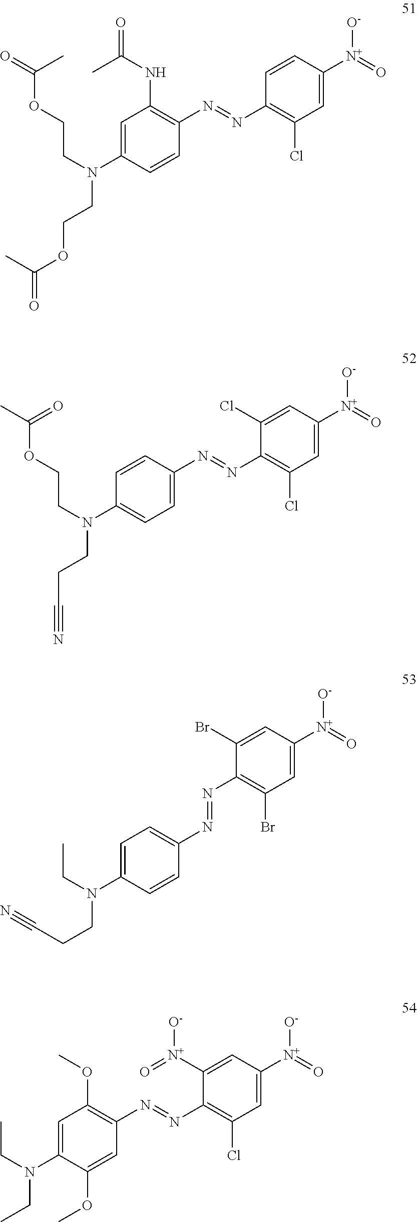

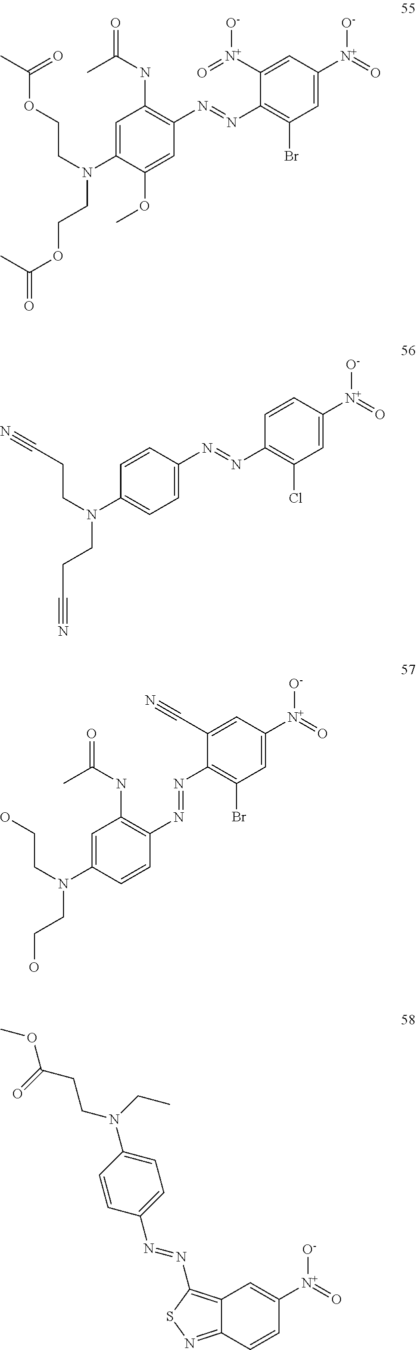

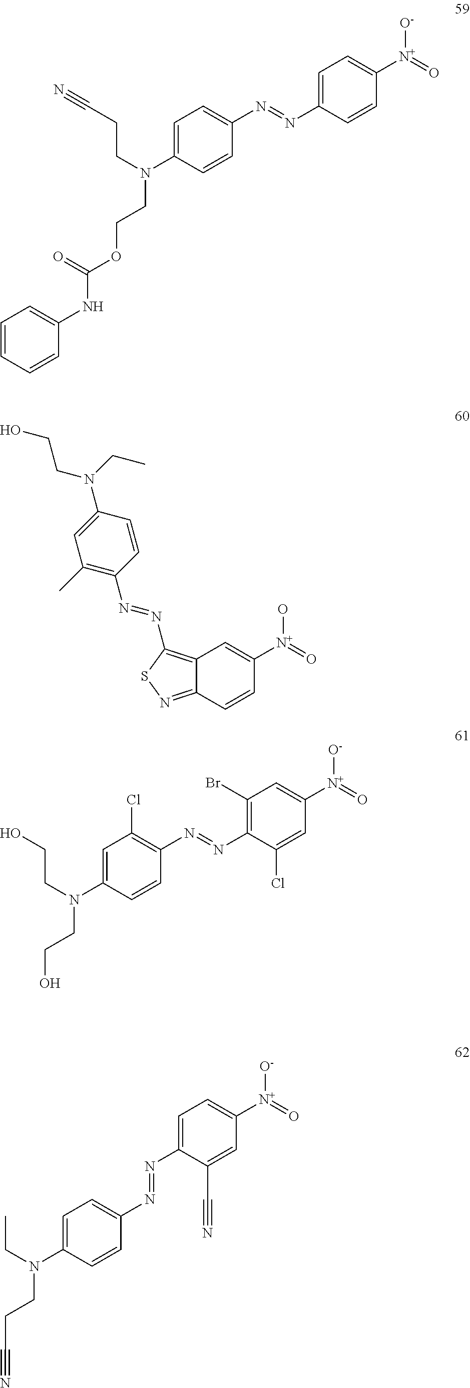

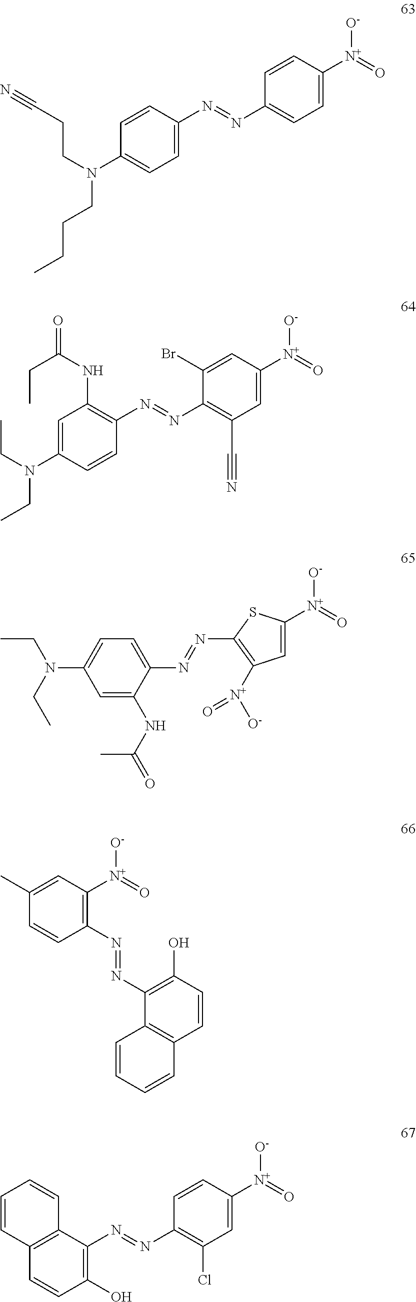

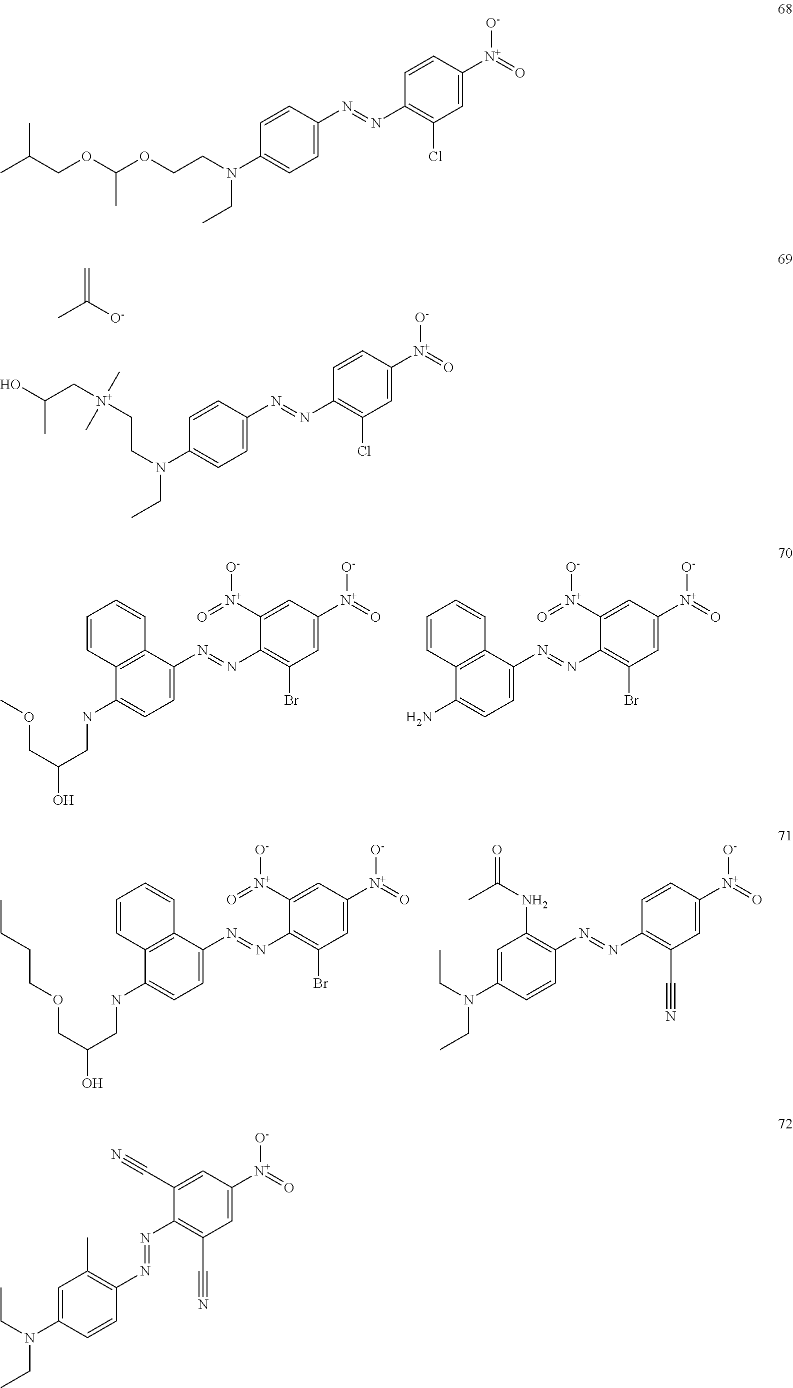

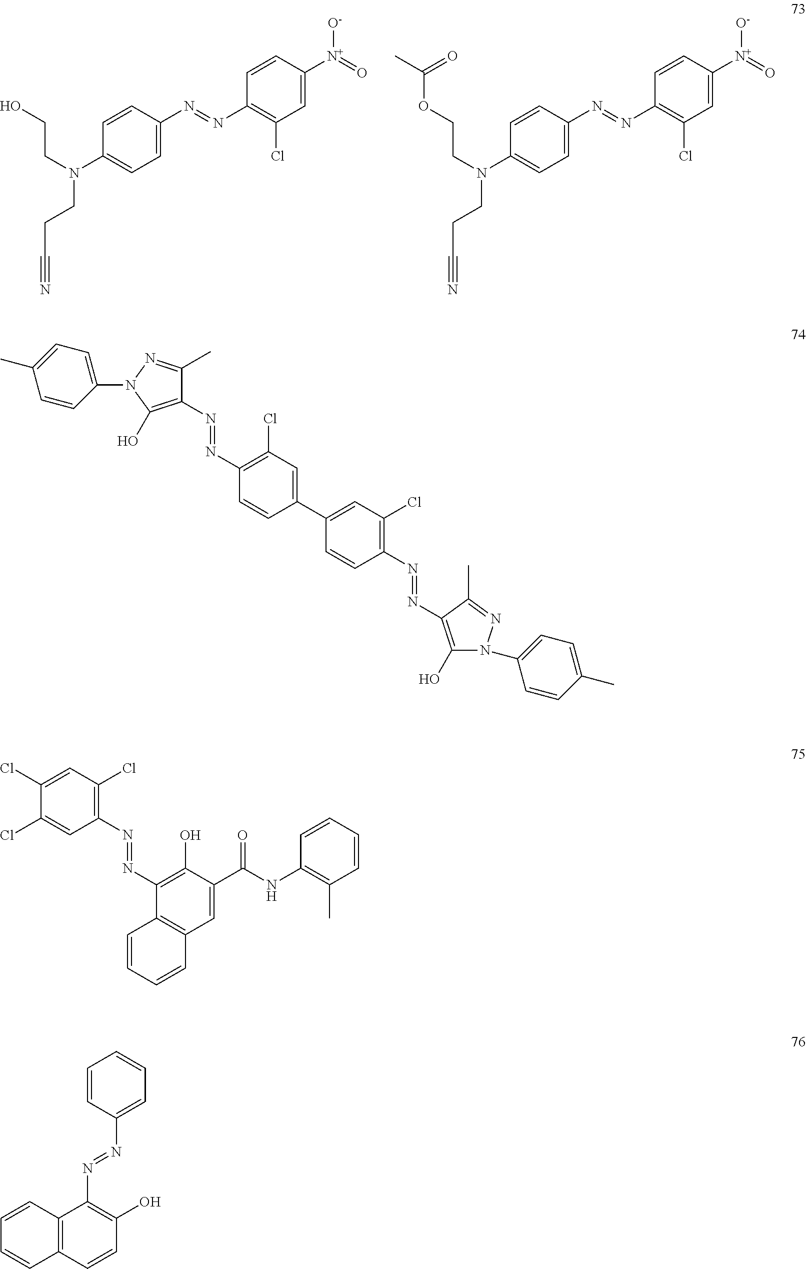

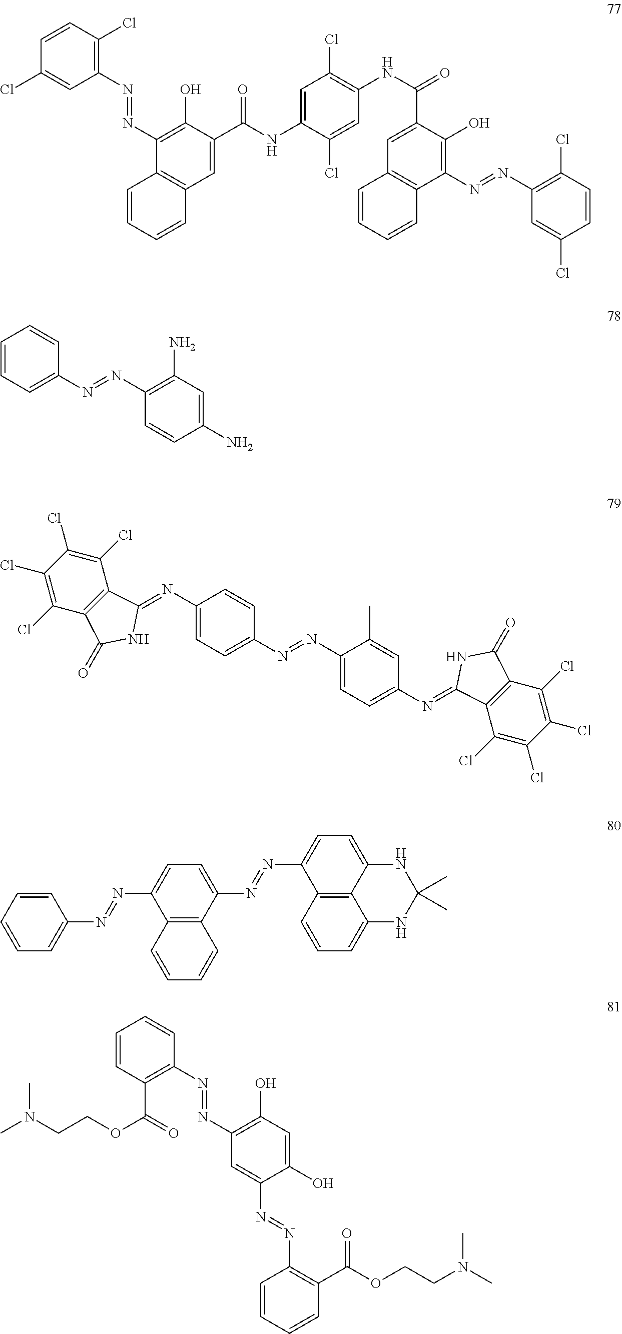

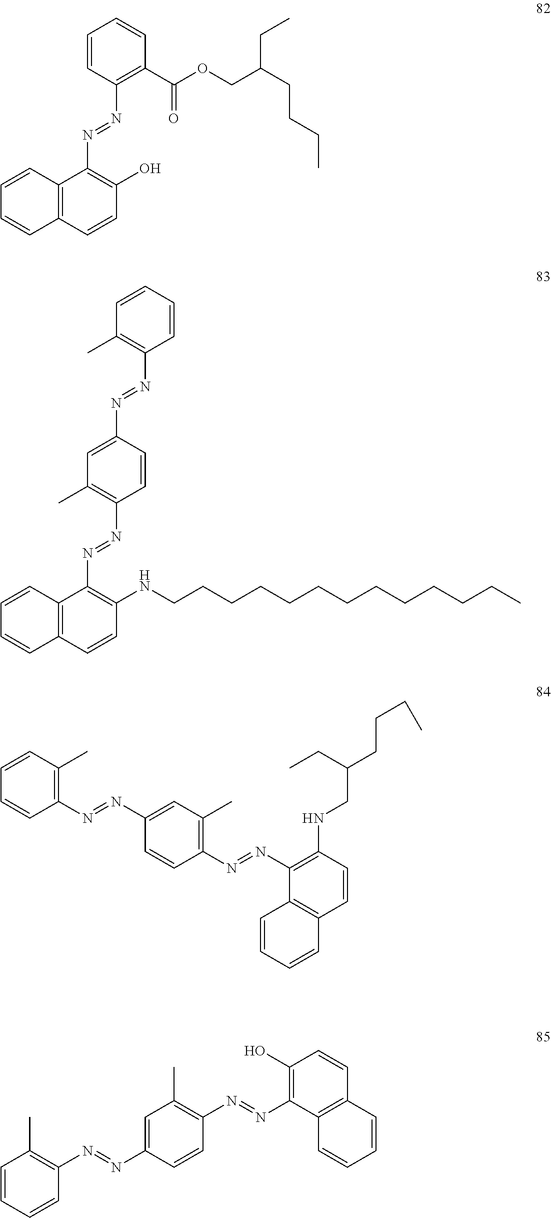

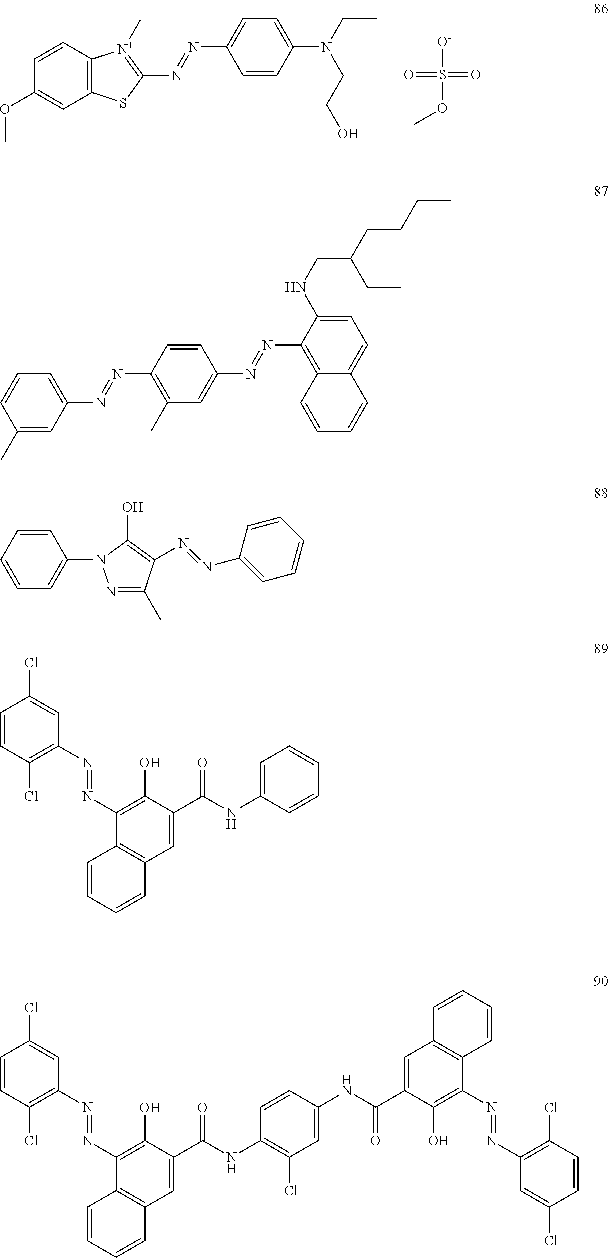

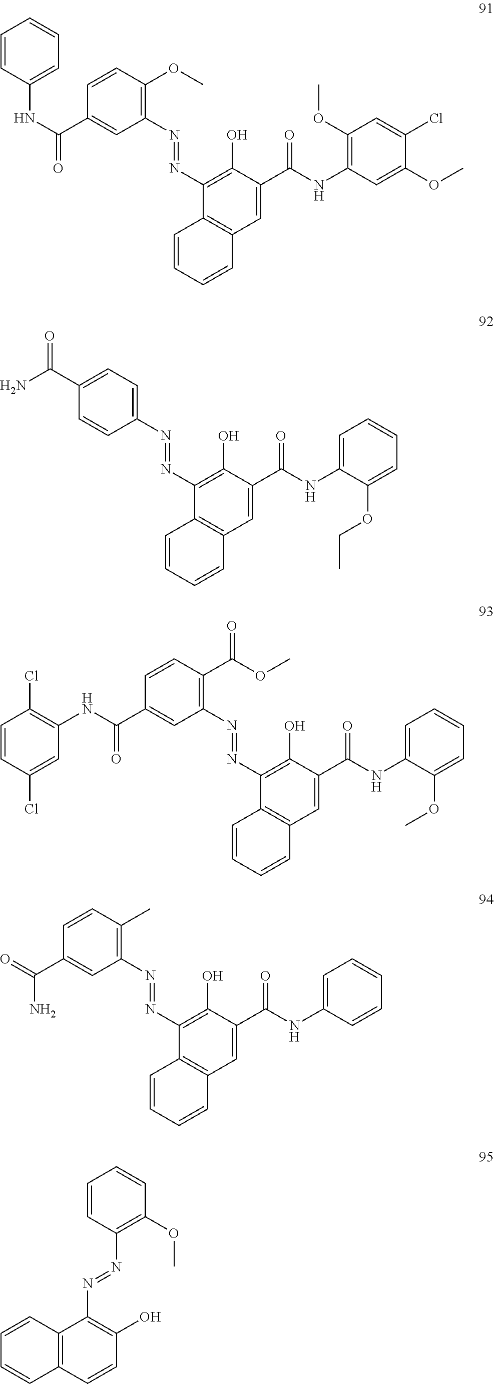

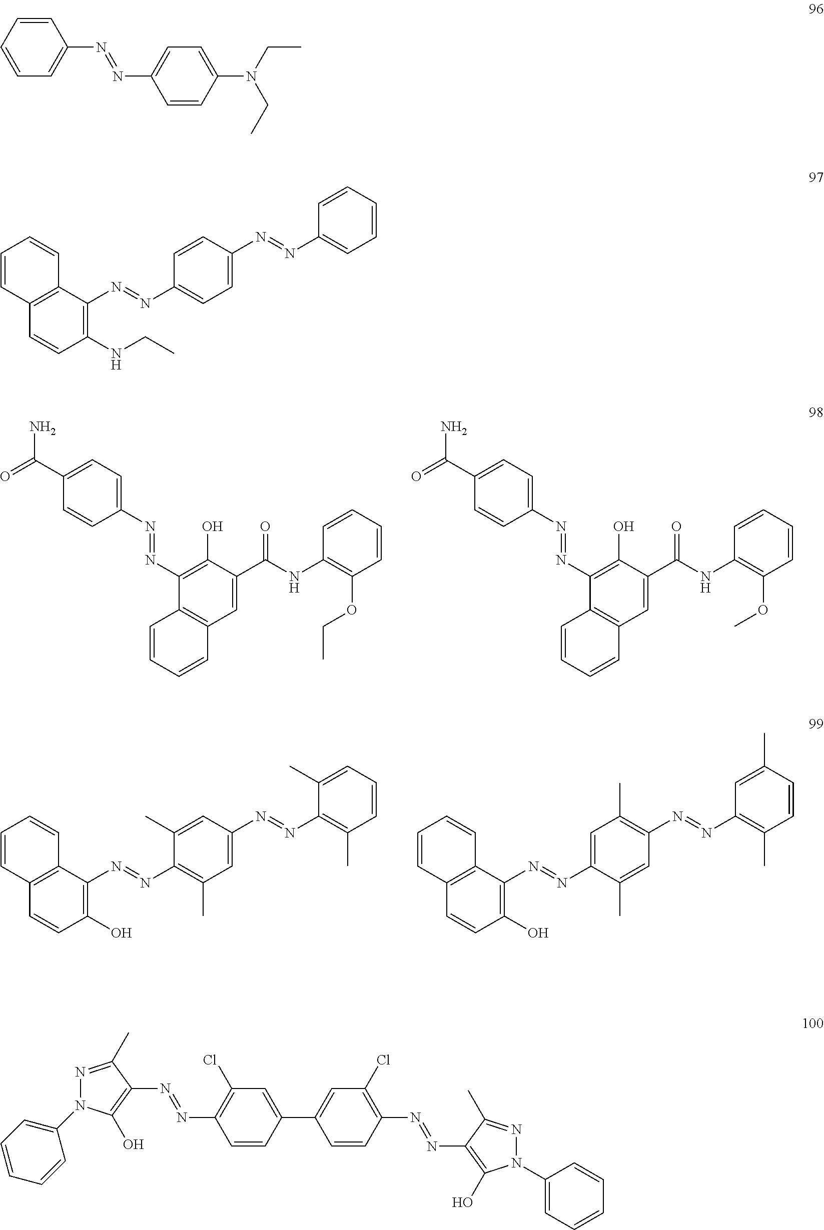

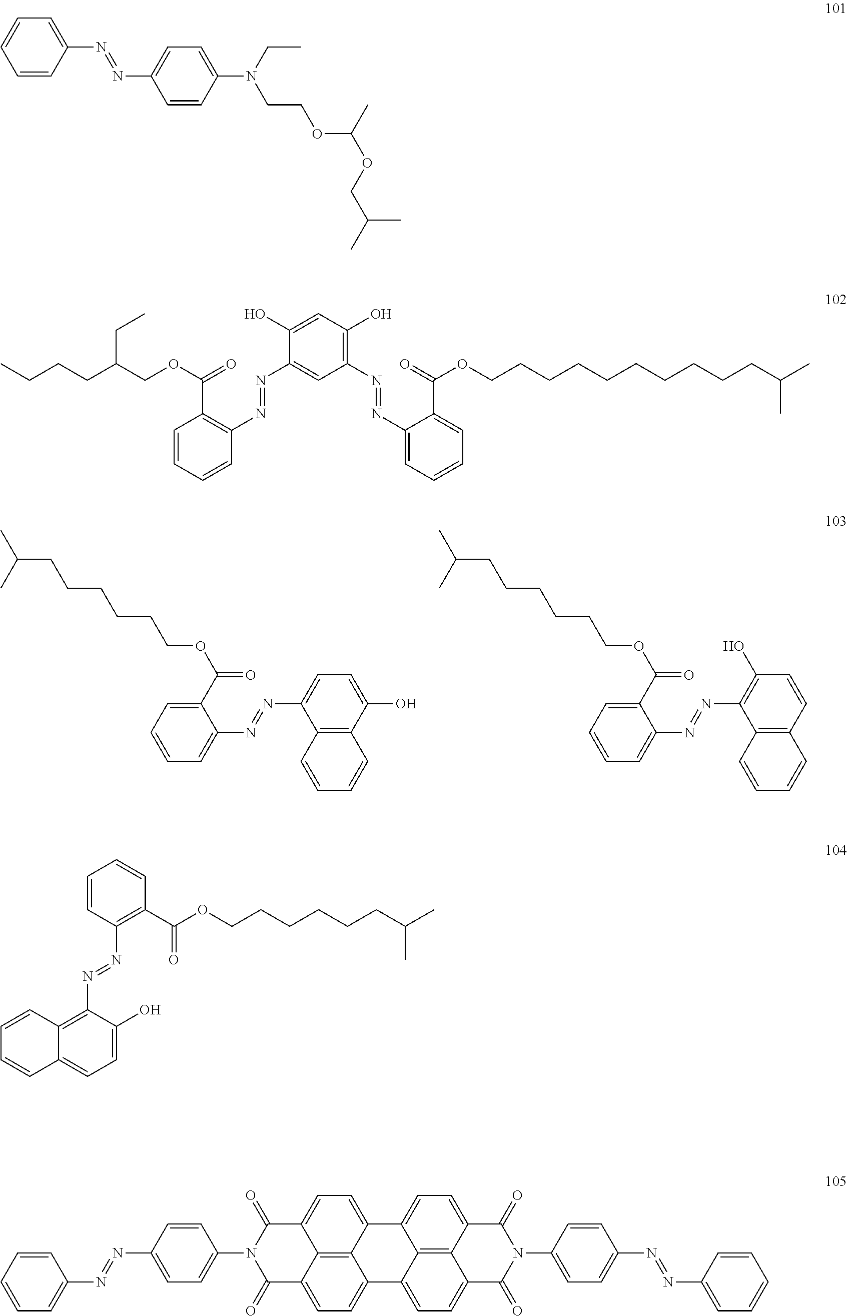

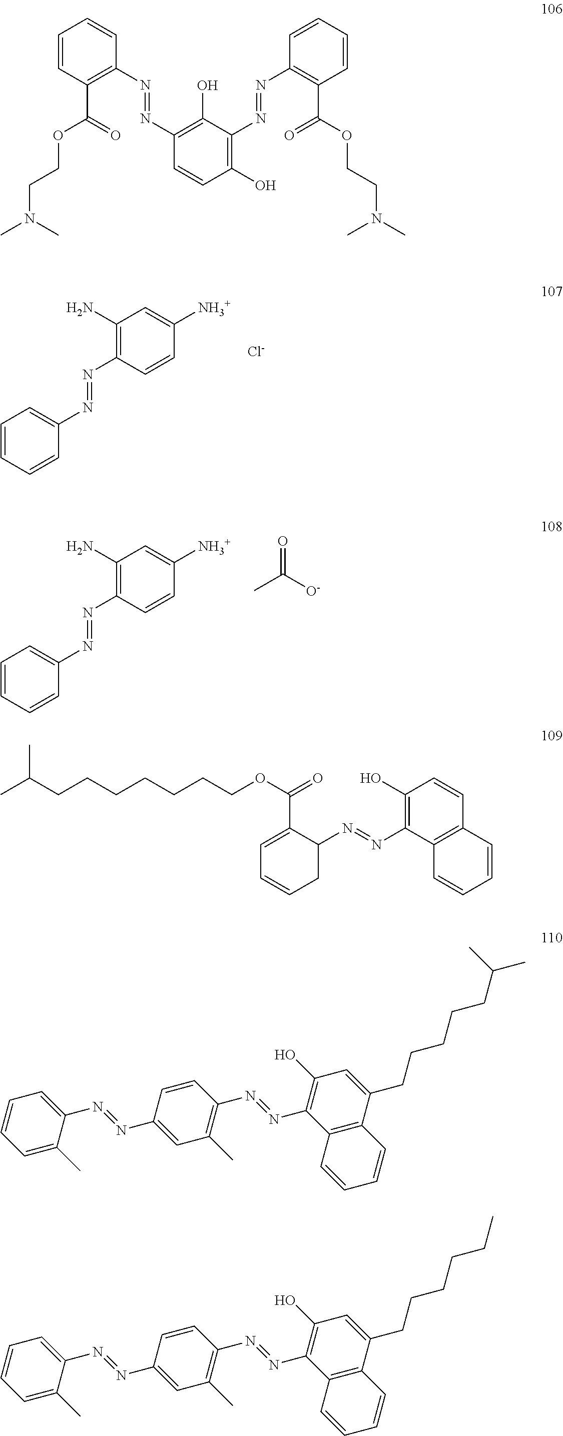

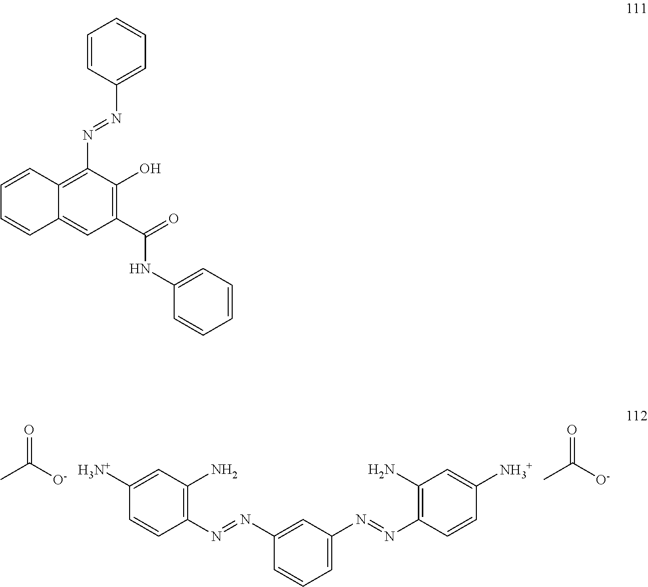

Preferably, the azo compound is selected from the group consisting of the azo compounds 1, 3, 5, 6, 30, 78, 59 and 112, and, in particular, 1 (=E123), 3 (=E110), 5 (=E128) and 6 (=E129):

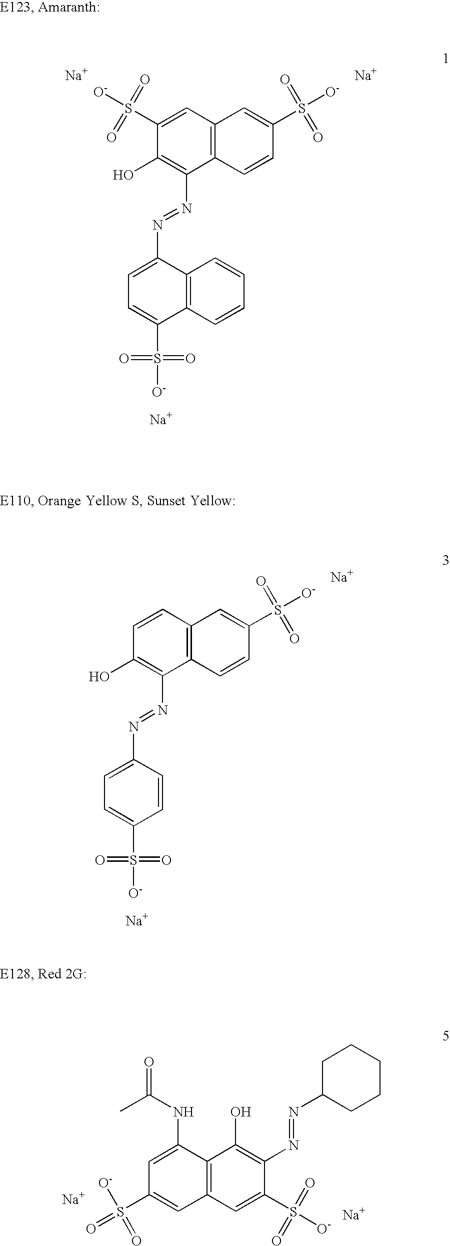

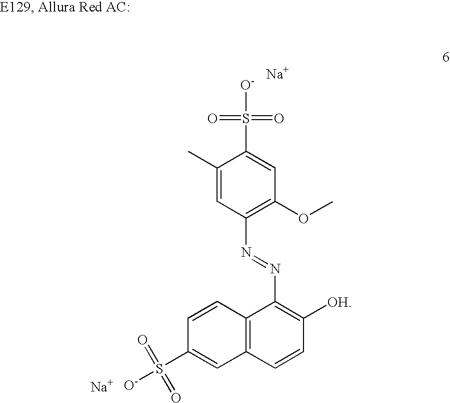

In this case the combinations hereinafter of two preferred azo compounds are particularly advantageous: E110/E128, E110/E129, E128/E129, E123/E110, E123/E128 and E123/E129, in particular E110/E129.

Preferably, these are ACK-sweetened drinks, preferably caffeine-comprising drinks, which, in addition to the carotenoid particles, comprise the azo compounds E 110 and E 129. These can comprise further HISs and are particularly preferably sugar-free and carbohydrate-free.

### EXAMPLES AND COMPARATIVE EXPERIMENTS

### Examples 1 to 18

### The Use of Water-Dispersible Carotenoid Nanoparticles for Taste Modulation of Sweeteners

For Examples 1 to 17, the substances hereinafter were used.

### Sweeteners:

Acesulfame K (ACK) from Fluca Bio Chemika;
Aspartame (ASP) from Fluca Bio Chemika;

### Formulations (A1) to (A3):

### Formulation (A11):

Components: Water-dispersible nanoparticles of a particle size of 100 to 300 nm determined using electron microscopy images, based on the formulation (A11), 10% by weight of beta-carotene nanoparticles in vegetable oil droplets, stabilized by DL-alpha-tocopherol (E 307) and embedded in a matrix of modified food starch (E 1450) and glucose syrup, and also tricalcium phosphate (E 341) as anticaking agent;
Production: For example using the continuous mixing chamber process described by Dieter Horn and Jens Rieger in “Organische Nanopartikel in wässriger Phase—Theorie, Experiment und Anwendung” [Organic nanoparticles in aqueous phase—theory, experiment and application], in Angewandte Chemie, 2001, volume 113, pages 4477, right-hand column, last paragraph, to 4478, right-hand column, first paragraph, and also in the section “4.1.2. Water-insoluble active components”, pages 4481 to 4483, and subsequent spray drying;

### Formulation (A12):

Components: Water-dispersible nanoparticles of a particle size of 100 to 300 nm determined using electron microscopy images, comprising, based on the Formulation (A12), 10% by weight of beta-carotene nanoparticles in vegetable oil droplets, stabilized by DL-alpha-tocopherol (E 307) and ascorbyl palmitate (E 304) and embedded in a matrix of fish gelatin (protective colloid) and glucose syrup, and also tricalcium phosphate (E 341) as anticaking agent;
Production: For example using the continuous mixing chamber method described by Dieter Horn and Jens Rieger in “Organische Nanopartikel in wässriger Phase—Theorie, Experiment und Anwendung” [Organic nanoparticles in aqueous phase—theory, experiment and application], in Angewandte Chemie, 2001, volume 113, pages 4477, right-hand column, last paragraph, to 4478, right-hand column, first paragraph, and also in the section “4.1.2. Water-insoluble active components”, pages 4481 to 4483, and subsequent spray drying;

### Formulation (A13):

Components: Water-dispersible nanoparticles of a median particle size of 100 to 300 nm determined using electron microscopy images, based on Formulation (A13), 10% by weight of lycopene nanoparticles in vegetable oil droplets, stabilized by DL-alpha-tocopherol (E 307) and embedded in a matrix of modified food starch (E 1450) and glucose syrup, and also tricalcium phosphate (E 341) as anticaking agent;
Production: For example using the continuous mixing chamber method described by Dieter Horn and Jens Rieger in “Organische Nanopartikel in wässriger Phase—Theorie, Experiment und Anwendung” [Organic nanoparticles in aqueous phase—theory, experiment and application], in Angewandte Chemie, 2001, volume 113, pages 4477, right-hand column, last paragraph, to 4478, right-hand column, first paragraph, and also in the section “4.1.2. Water-insoluble active components”, pages 4481 to 4483, and subsequent spray drying;

### Formulation (A14):

Components: Water-dispersible nanoparticles of a particle size of 200 to 300 nm determined using electron microscopy images; comprising, based on the formulation (A14), 10% by weight of beta-carotene nanoparticles in corn germ oil droplets, embedded in a matrix of modified food starch;
Production, for example using the process described in the textbook by J. C. Bauernfeind, “Carotenoids as Colorants and Vitamin A Precursors. Technological and Nutritional Applications”, Chapter 2, J. C. Bauernfeind and H. Kläui, “Carotenoids as Food Color”, pages 92 to 95, Academic Press, ISBN 0-12-082850-2, 1981, and subsequent spray drying;

### Formulation (A2):

O/W microemulsion of a droplet size of 200 nm determined using quasielastic light scattering, comprising, based on the formulation (A2), 10% by weight of beta-carotene dissolved in vegetable oil and triglycerides of fatty acids of medium chain length, stabilized by DL-alpha-tocopherol (E 307) and ascorbyl palmitate (E 304) and emulsified in a glycerol/water mixture;

### Formulation (A3):

Components: Water-dispersible particles having particle sizes between 150 and 850 μm; comprising, based on the formulation (A3), 10% by weight of beta-carotene nanoparticles, modified starch, sucrose, DL-alpha-tocopherol (E 307), ascorbic acid, sodium ascorbate and tricalcium phosphate (E 341);
Production, for example, by grinding in suspension, and subsequent spray drying of the resultant particles.

### Azo Compounds:

No. 3: E110, Sunset Yellow from Sigma;
No. 6: E129, Allura Red from Sigma;

### Cola Drink:

What was termed “zero cola” without sweetener was produced as follows:
36 g of cola flavor (cola base from Döhler, Darmstadt, article no. 200380),
7.7 g of orthophosphoric acid 85% ultrapure (Karl Roth GmbH+Co KG, Karlsruhe, article no. 9079.1),
3.6 g of citric acid 99.5% p.a. (Karl Roth GmbH+Co KG, Karlsruhe, article no. 3958.2),
2.4 g of sodium benzoate from Fluka, Sigma-Aldrich, Steindrunn, and
1.2 g of caffeine, anhydrous, 99% from Fluka, Sigma-Aldrich, Steindrunn
   were dissolved in 600 ml of tap water. 50 ml portions of this concentrate were made up in each case to one liter of “zero cola”.

### Quantitative Sensory Testing—General Protocol:

Consensus profiles of samples 1 to 18 of Examples 1 to 18 and also of control samples 1 to 6 were prepared in agreement with DIN 10967-2/ISO 11035. For this, 8 trained testers which had been selected in accordance with the DIN/ISO provisions were made familiar with the products by definition and training of the predetermined feature properties.

Subsequently, the testers tasted the samples 1 to 17 in order to assess the taste, aftertaste and mouth feel in accordance with the given feature properties. The respective consensus profiles were summarized by the test leader in the form of tables and spider's web diagrams. Hereinafter, for the sake of clarity, only the tables are reproduced.

### Samples 1 to 18 and Control Samples 1 to 5—Composition of Matter:

Samples 1 to 18 and the control samples 1 to 5 had the compositions of matter described hereinafter. The respective abbreviations which are used in the tables hereinafter are given in brackets.

### Control Sample 1:

Water+500 mg/l of ACK (abbreviation: water/ACK)

### Control Sample 2:

Water+350 mg/l of ASP (abbreviation: water/ASP)

### Control Sample 3:

Water+140 mg/l of ACK+350 mg/l of ASP (abbreviation: water/ACK/ASP)

### Control Sample 4:

Zero cola+500 mg/l of ACK (abbreviation: cola/ACK)

### Control Sample 5:

Zero cola+140 mg/l of ACK+350 mg/l of ASP (abbreviation: cola/ACK/ASP)

### Sample 1—Example 1

Water+500 mg/l of ACK+formulation (A11), corresponding to 1 ppm of beta-carotene (abbreviation: water/ACK/A11)

### Sample 2—Example 2:

Water+500 mg/l of ACK+formulation (A13), corresponding to 1 ppm of lycopene (abbreviation: water/ACK/A13)

### Sample 3—Example 3:

Water+500 mg/l of ACK+formulation (A12), corresponding to 1 ppm of beta-carotene (abbreviation: water/ACK/A12)

### Sample 4—Example 4:

Water+500 mg/l of ACK+formulation (A11), corresponding to 1 ppm of beta-carotene, +2.3 mg/l of azo compound E10+azo compound 2.5 mg/l of E129
(abbreviation: water/ACK/A11/E110/E129)

### Sample 5—Example 5:

Water+140 mg/l of ACK+350 mg/l of ASP+formulation (A11), corresponding to 1 ppm of beta-carotene
(abbreviation: water/ACK/ASP/A11)

### Sample 6—Example 6:

Water+350 mg/l of ASP+formulation (A11), corresponding to 1 ppm of beta-carotene
(abbreviation: water/ASP/A11)

### Sample 7—Example 7:

Water+140 mg/l of ACK+350 mg/l of ASP+formulation (A12), corresponding to 1 ppm of beta-carotene
(abbreviation: water/ACK/ASP/A12)

### Sample 8—Example 8:

Zero cola+500 mg/l of ACK+formulation (A11), corresponding to 1 ppm of beta-carotene
(abbreviation: cola/ACK/A11)

### Sample 9—Example 9:

Zero cola+140 mg/l of ACK+350 mg/l of ASP+formulation (A11), corresponding to 1 ppm of beta-carotene
(abbreviation: cola/ACK/ASP/A11)

### Sample 10—Example 10:

Zero cola+140 mg/l of ACK+350 mg/l of ASP+formulation (A12), corresponding to 1 ppm of beta-carotene
(abbreviation: cola/ACK/ASP/A12)

### Sample 11—Example 11:

Zero cola+500 mg/l of ACK+formulation (A11), corresponding to 1 ppm of beta-carotene, +2.5 mg/l of azo compound E129
(abbreviation: cola/ACK/A11/E129)

### Sample 12—Example 12:

Zero cola+500 mg/l of ACK+formulation (A11), corresponding to 1 ppm of beta-carotene, +2.3 mg/l of azo compound E110
(abbreviation: cola/ACK/A11/E110)

### Sample 13—Example 13:

Zero cola+500 mg/l of ACK+formulation (A11), corresponding to 1 ppm of beta-carotene, +2.3 mg/l of azo compound E10+2.5 mg/l of azo compound E129
(abbreviation: cola/ACK/A11/E110)

### Sample 14—Example 14:

Zero cola+140 mg/l of ACK+350 mg/l of ASP+formulation (A11), corresponding to 1 ppm of beta-carotene, +2.3 mg/l of azo compound E110
(abbreviation: cola/ACK/ASP/A11/E110)

### Sample 15—Example 15:

Zero cola+140 mg/l of ACK+350 mg/l of ASP+formulation (A11), corresponding to 1 ppm of beta-carotene, +2.3 mg/l of azo compound E110+2.5 mg/l of azo compound E129
(abbreviation: cola/ACK/ASP/A11/E110/E129)

### Sample 16—Example 16:

Water+500 mg/l of ACK+formulation (A14), corresponding to 1 ppm of beta-carotene
(abbreviation: water/ACK/A14)

### Sample 17—Example 17:

Water+500 mg/l of ACK+formulation (A2), corresponding to 1 ppm of beta-carotene
(abbreviation: water/ACK/A2)

### Sample 18—Example 18:

Water+500 mg/l of ACK+formulation (A3), corresponding to 1 ppm of beta-carotene
(abbreviation: water/ACK/A3)

### Samples 1 to 7 and Also 16 to 18 and Control Samples 1 to 3—Experimental Results:

The results of the quantitative sensory testing of samples 1 to 7 and 16 to 18, and also of control samples 1 to 3, are summarized in Table 1. The measurement 0 means that the relevant sensory property was not present; the measurement 10 means that the relevant sensory property was strongly present.

TABLE 1
Quantitative sensory testing of samples 1 to 7 and 16 to 18, and also
control samples 1 to 3 - consensus profile

| Control sample/ | Taste | | Mouth feel | | | | Aftertaste | | | |
|---|---|---|---|---|---|---|---|---|---|---|
| sample | S^{a)} | Bt^{b)} | C^{c)} | K^{d)} | Bl^{e)} | A^{f)} | S(N)^{g)} | Bt(N)^{h)} | A(N)ⁱ⁾ | C(N)^{j)} |
| Control sample 1 | 6 | 4 | 4 | 0 | 4 | 2 | 3 | 6 | nd^{k)} | 0 |
| Water/ACK | | | | | | | | | | |
| Control sample 2 | 3^{l)} | 1 | 0 | 0 | 1 | 2 | 1^{m)} | 0 | 2 | nd^{k)} |
| Water/ASP | | | | | | | | | | |
| Control sample 3 | 8 | 0 | 2 | 0 | 2 | 1 | 6 | 0 | 0 | nd^{k)} |
| Water/ACK/ASP | | | | | | | | | | |
| Sample 1 | 8 | 2 | 3 | 0 | 1 | 4 | 4 | 0 | nd^{k)} | nd^{k)} |
| Water/ACK/A11 | | | | | | | | | | |
| Sample 2 | 7 | 5 | 5 | 1 | 1 | 3 | 3 | 1 | nd^{k)} | nd^{k)} |
| Water/ACK/A13 | | | | | | | | | | |
| Sample 3 | 5 | 3 | 4 | 0 | 6 | 4 | 2 | 0 | nd^{k)} | 0 |
| Water/ACK/A12 | | | | | | | | | | |
| Sample 4 | 7 | 0 | 2 | 0 | 1 | 3 | 3 | 0 | nd^{k)} | nd^{k)} |
| Water/ACK/A12/ | | | | | | | | | | |
| E110/E129 | | | | | | | | | | |
| Sample 5 | 9 | 0 | 1 | 0 | 2 | 1 | 5 | 0 | 3 | nd^{k)} |
| Water/ACK/ | | | | | | | | | | |
| ASP/A11 | | | | | | | | | | |
| Sample 6 | 3 | 0 | 0 | 0 | 2 | 2 | 3 | 0 | 2 | nd^{k)} |
| Water/ASP/A11 | | | | | | | | | | |
| Sample 7 | 8 | 0 | 3 | 0 | 2 | 1 | 6 | 0 | 0 | nd^{k)} |
| Water/ACK/ | | | | | | | | | | |
| ASP/A12 | | | | | | | | | | |
| Sample 16 | 4 | 1 | 4 | 0 | 2 | 3 | 2 | 1 | nd^{k)} | nd^{k)} |
| Water/ACK/A14 | | | | | | | | | | |
| Sample 17 | 6 | 4 | 4 | 0 | 1 | 2 | 3 | 1 | nd^{k)} | nd^{k)} |
| Water/ACK/A2 | | | | | | | | | | |
| Sample 18 | 5 | 4 | 3 | 0 | 1 | 2 | 3 | 5 | nd^{k)} | nd^{k)} |
| Water/ACK/A3 | | | | | | | | | | |

^{a)}S = sweet;
^{b)}Bt = bitter;
^{c)}C = chemical;
^{d)}K = prickly;
^{e)}Bl = coating;
^{f)}A = drying;
^{g)}S(N) = sweet aftertaste;
^{h)}Bt(N) = bitter aftertaste;
ⁱ⁾A(N) = drying out in aftertaste;
^{j)}C(N) = chemical aftertaste;
^{k)}nd = not determined;
^{l)}first aqueous, then sweet aftertaste;
^{m)}aftertaste decays rapidly;

From the results of Table 1, the following taste-modulating effects were found for samples 1 to 7 in detail:

### Sample 1:

The formulation (A11) reduced the bitter taste and completely reduced the bitter aftertaste of ACK in water.

### Sample 2:

The formulation (A13) reduced the bitter aftertaste of ACK in water.

### Sample 3:

The formulation (A12) completely reduced the bitter aftertaste of ACK in water; the bitter taste was slightly reduced. However, sample 3 was perceived to be somewhat more strongly coating and somewhat less sweet than sample 1.

### Sample 4:

The triple combination formulation (A11)/E110/E129 showed synergistic effects in the modulation of the bitter taste and aftertaste. Not only the bitter taste but also the bitter aftertaste were completely reduced.

### Sample 5:

The formulation (A11) only very slightly affected the taste and aftertaste of ACK/ASP in water.

### Sample 6:

The formulation (A11) caused scarcely any taste changes of ASP in water. However, the slight bitter taste of ASP in water was no longer perceived.

### Sample 7:

The formulation (A12) affected only to a very slight extent the taste and aftertaste of ACK/ASP in water.

### Sample 16:

The formulation (A14) reduced the bitter taste of ACK in water and completely reduced the bitter aftertaste. In addition, sample 16 was less coating than control sample 1.

### Sample 17:

The formulation (A2) reduced the bitter taste of ACK in water and completely reduced the bitter aftertaste. In addition, sample 17 was significantly less coating than control sample 1.

### Sample 18:

The formulation (A3) somewhat reduced the bitter aftertaste of ACK in water. In addition, sample 18 tasted significantly less chemical and was significantly less coating than control sample 1.

Overall, using the formulations (A11) to (A14) and also (A2) and (A3), in particular in combination with the azo compounds E110 and E129, the bitter taste and the bitter aftertaste of ACK in water were significantly reduced.

### Samples 8 to 15 and Control Samples 4 and 5—Experimental Results:

The results of the quantitative sensory testing of samples 8 to 15 and control samples 4 and 5 are summarized in Table 2. The measurement 0 means that the relevant sensory property was not present; the measurement 10 denotes that the relevant sensory property was strongly present.

TABLE 2
Quantitative sensory testing of samples 8 to 15 and
control samples 4 and 5 - consensus profiles

| Control probe/ | Taste | | | | | Mouth feel | | | | Aftertaste | | | |
|---|---|---|---|---|---|---|---|---|---|---|---|---|---|
| Probe | BS^{a)} | Sä^{b)} | Bt^{c)} | KC^{d)} | M^{e)} | Co^{f)} | S^{g)} | A^{h)} | Blⁱ⁾ | Au^{j)} | S(N)^{k)} | Bt(N)^{l)} | K(N)^{m)} |
| Control sample 4 | 1 | 6 | 4 | 4 | 2 | 4 | 4 | 2 | 4 | 3 | 3 | 1 | 0 |
| cola/ACK | | | | | | | | | | | | | |
| Control sample 5 | 7 | 4 | 1ⁿ⁾ | 4 | 2 | 5 | 6 | 0 | 1 | 2 | 6 | 0 | 2 |
| cola/ACK/ASP | | | | | | | | | | | | | |
| Sample 8 | 1 | 6 | 1 | 4 | 3 | 3 | 3 | 1 | 3 | 3 | 3 | 1 | 2 |
| cola/ACK/A11 | | | | | | | | | | | | | |
| Sample 9 | 8 | 3 | 0 | 4 | 2 | 4 | 9 | 1 | 1 | 3 | 5 | 0 | 2 |
| cola/ACK/ | | | | | | | | | | | | | |
| ASP/A11 | | | | | | | | | | | | | |
| Sample 10 | 6 | 5 | 0 | 4 | 1 | 4 | 5 | 0 | 1 | 1 | 5 | 0 | 1 |
| cola/ACK/ | | | | | | | | | | | | | |
| ASP/A12 | | | | | | | | | | | | | |
| Sample 11 | 0 | 3ⁿ⁾ | 0 | 5 | 4 | 2 | 2 | 0 | 1 | 1 | 2 | 0 | 0 |
| cola/ACK/A11/ | | | | | | | | | | | | | |
| E129 | | | | | | | | | | | | | |
| Sample 12 | 2 | 5 | 0 | 4 | 2 | 3 | 4 | 0 | 1 | 3 | 3 | 0 | 0 |
| cola/ACK/A11/ | | | | | | | | | | | | | |
| E110 | | | | | | | | | | | | | |
| Sample 13 | 3 | 4 | 0 | 3 | 1 | 5 | 5 | 0 | 1 | 2 | 4 | 0 | 0 |
| cola/ACK/A11/ | | | | | | | | | | | | | |
| E129/E110 | | | | | | | | | | | | | |
| Sample 14 | 5 | 3 | 0 | 2 | 0 | 5 | 5 | 0 | 1 | 1 | 5 | 0 | 0 |
| cola/ACK/ASP/ | | | | | | | | | | | | | |
| A11/E110 | | | | | | | | | | | | | |
| Sample 15 | 7 | 3 | 0 | 2 | 0 | 5 | 6 | 0 | 1 | 1 | 6 | 0 | 0 |
| cola/ACK/ASP/ | | | | | | | | | | | | | |
| A11/E129/E110 | | | | | | | | | | | | | |

^{a)}BS = beginning of sweetness;
^{b)}Sä = acidic;
^{c)}Bt = bitter;
^{d)}KC = artificial/chemical;
^{e)}M = metallic;
^{f)}Co = cola;
^{g)}S = sweet;
^{h)}A = astringent;
ⁱ⁾Bl = coating;
^{j)}Au = drying;
^{k)}S(N) = sweet aftertaste;
^{l)}Bt(N) = bitter aftertaste;
^{m)}K(N) = prickly aftertaste
ⁿ⁾only on swallowing for about 10 seconds;

From the results of Table 2, in detail, the following taste-modulating effects were found for samples 8 to 15.

### Sample 8:

The formulation (A11) had a beneficial effect on the modulation of the bitter taste. However, the cola flavor decreases slightly and the sample has a prickly aftertaste.

### Sample 9:

The formulation (A11) only slightly affected the taste of ACK/ASP in cola. Sample 9 was somewhat less acidic and not bitter. It had somewhat less cola flavor but somewhat more sweetness.

### Sample 10:

The formulation (A12) only slightly affected the taste of ACK/ASP in cola. However, the bitter taste was no longer present.

### Sample 11:

The formulation (A11) and E129 completely reduced the bitter taste and bitter aftertaste of ACK in cola. The sweet taste and the sweet aftertaste were somewhat reduced.

### Sample 12:

The formulation (A11) and E110 completely reduced the bitter taste and bitter aftertaste of ACK in cola. Sample 12, in addition, was found to be non astringent. The cola flavor was only somewhat reduced.

### Sample 13:

The formulation (A11), E110 and E129 completely reduced the bitter taste and aftertaste of ACK in cola. The other adverse taste attributes (acidic, artificial/chemical, metallic, astringent, coating, drying) were reduced, whereas the favorable taste attributes (sweetness, cola) were improved.

### Sample 14:

The formulation (A11) and E110 somewhat reduced the acidic, bitter, artificial/chemical and metallic taste, and also the prickly aftertaste of ACK/ASP in cola. The sweetness was no longer so intensely pronounced as in control sample 6.

### Sample 15:

The formulation (A11), E110 and E129 somewhat reduced the acidic, bitter, artificial/chemical and metallic taste, and also the prickly aftertaste of ACK/ASP in cola. In addition, sample 15 was less drying than control sample 6. The bitter aftertaste was not reduced. Otherwise, the positive taste attributes (sweetness, cola) were not affected.

### Comparative Experiments C1 and C2

The use of carotenoid-free placebos C2 and C1 for taste modulation of sweeteners

Example 1 was repeated, except that, instead of the formulation (A11)
in comparison experiment C1, a carotenoid-free placebo C1, which had been produced from modified starch, tocopherol, vegetable oil and glucose by the continuous mixing chamber process described by Dieter Horn and Jens Rieger in “Organische Nanopartikel in wässriger Phase—Theorie, Experiment und Anwendung” [Organic nanoparticles in aqueous phase—theory, experiment and application], in Angewandte Chemie, 2001, volume 113, pages 4477, right-hand column, last paragraph, to 4478, right-hand column, first paragraph, and also in the section “4.1.2. In Wasser unlösliche Wirkstoffe” [Water-insoluble active compounds”], pages 4481 to 4483, and spray drying (abbreviation: water/ACK/C1), and
in comparative experiment C2, a carotenoid-free placebo C2, which had been produced by mixing modified starch, tocopherol, vegetable oil and glucose and spray drying the resulting mixture (abbreviation: water/ACK/C2),
   were used.

The results of quantitative sensory testing of samples C1 and C2 of the comparative experiments C1 and C2 are compared in Table 3 with the results obtained with control sample 1. The measurements have the meaning reported above for Tables 1 and 2.

TABLE 3
Quantitative sensory testing of samples C1 and C2
and control sample 1 - consensus profiles

| Control sample/ | Taste | | Mouth feel | | | | Aftertaste | |
|---|---|---|---|---|---|---|---|---|
| sample | S^{a)} | Bt^{b)} | C^{c)} | K^{d)} | Bl^{e)} | A^{f)} | S(N)^{g)} | Bt(N)^{h)} |
| Control sample 1 | 6 | 4 | 4 | 0 | 4 | 2 | 3 | 6 |
| water/ACK | | | | | | | | |
| Sample C1 | 8 | 6 | 4 | 0 | 2 | 2 | 5 | 2 |
| water/ACK/C1 | | | | | | | | |
| Sample C3 | 7 | 5 | 4 | 0 | 4 | 3.5 | 4 | 1 |
| water/ACK/C2 | | | | | | | | |

^{a)}S = sweet;
^{b)}Bt = bitter;
^{c)}C = chemical;
^{d)}K = prickly;
^{e)}Bl = coating;
^{f)}A = drying;
^{g)}S(N) = sweet aftertaste;
^{h)}Bt(N) = bitter aftertaste;

Comparative experiments C1 and C2 showed that the beta-carotene-free placebos C1 and C2 somewhat increased the bitter taste of ACK in water, whereas they reduced the bitter aftertaste.

1-51. (canceled)

52. A composition comprising at least one water-dispersible carotenoid nanoparticle as a taste modulator.

53. The composition of claim 52, wherein said at least one water-dispersible carotenoid nanoparticle has a particle size of less than 1 μm.

54. The composition of claim 52, wherein said at least one water-dispersible carotenoid nanoparticle is liquid or solid.

55. The composition of claim 54, wherein the carotenoid or carotenoids in said at least one solid water-dispersible carotenoid nanoparticle are X-ray amorphous.

56. The composition of claim 54, wherein the carotenoid or carotenoids in said at least one liquid water-dispersible carotenoid nanoparticle are dissolved in a liquid medium or are present in the form of a solubilized mixed micelle.

57. The composition of claim 52, wherein said at least one water-dispersible carotenoid nanoparticle is present in a liquid or solid formulation comprising at least one additive permitted under food law and/or drugs law.

58. The composition of claim 57, wherein said at least one additive is selected from the group consisting of protective colloids, stabilizers against oxidative breakdown, emulsifiers, oils, plasticizers, compositions against caking, and polyols.

59. The composition of claim 57, wherein said at least one water-dispersible carotenoid nanoparticle is present in a solid formulation comprising at least one additive permitted under food law and/or drugs law, wherein said at least one water-dispersible carotenoid is embedded in a matrix of at least one additive.

60. The composition of claim 57, wherein said liquid or solid formulation comprises from 0.5 to 30% by weight of carotenoid or carotenoids, based on the total weight of said liquid or solid formulation.

61. The composition of claim 57, wherein said liquid or solid formulation comprises from 10 to 80% by weight of a protective colloid, based on the total weight of said liquid or solid formulation.

62. The composition of claim 57, wherein said liquid or solid formulation comprises from 20 to 70% by weight of plasticizer, based on on the total weight of said liquid or solid formulation.

63. The composition of claim 52, wherein said at least one water-dispersible carotenoid nanoparticle reduces the bitter taste and the bitter aftertaste of said composition.

64. The composition of claim 52, wherein said composition is a food, drink, article consumed for pleasure, sweetening agent, animal feed, cosmetic, or pharmaceutical.

65. The composition of claim 52, wherein said composition is a low-sugar composition having less than 10 g of sugar per liter or kg of said composition.

66. The composition of claim 52, wherein said composition comprises at least one High Intensity Sweetener (HIS) as a sweetening agent.

67. The composition of claim 52, wherein said at least one water-dispersible carotenoid nanoparticle is present in an amount such that a carotenoid concentration in the range of from 0.1 to 100 ppm results.

68. The composition of claim 52, wherein said at least one water-dispersible carotenoid nanoparticle is present in an amount such that a carotenoid concentration in the range of from 0.1 to 100 mg/l results.

69. The composition of claim 52, wherein said composition comprises at least one azo compound comprising at least one azo group, wherein said at least one azo compound reduces the bitter taste and aftertaste of said composition.

70. The composition of claim 69, wherein said composition comprises from 0.1 to 100 ppm of said at least one azo compound based on the total weight of said composition.

71. The composition of claim 69, wherein said at least one azo compound is selected from the group consisting of E110, E123, E128, and E129:

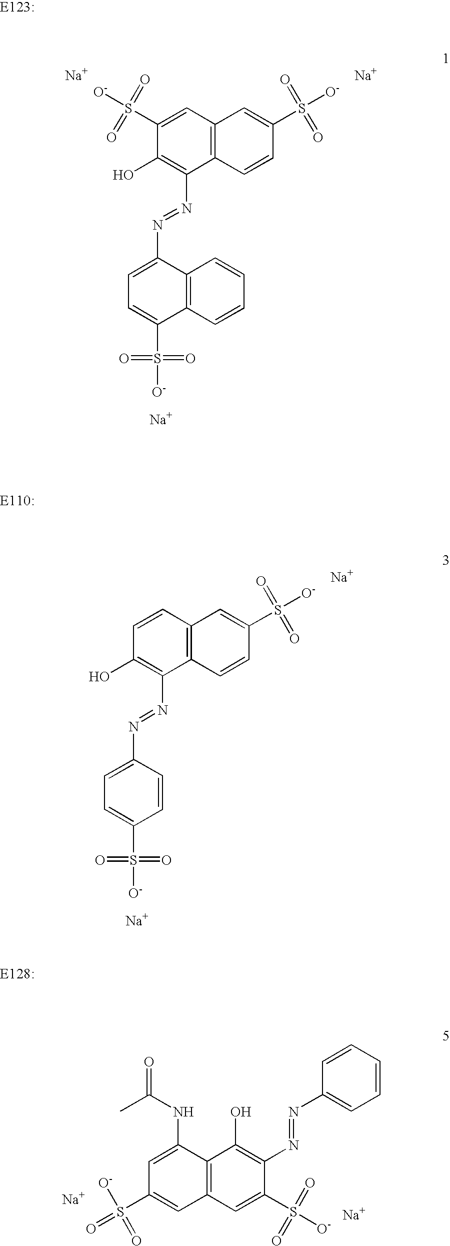

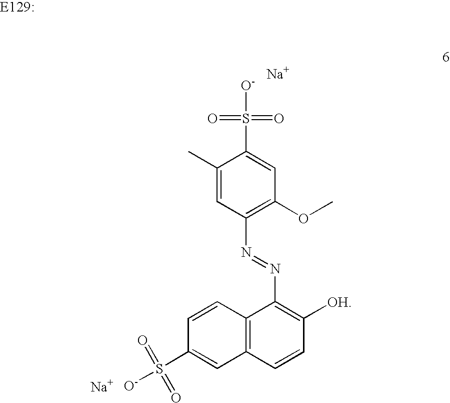

72. The composition of claim 66, wherein said at least one HIS is selected from the group consisting of acesulfame-potassium, aspartame, saccharin and salts thereof, cyclamate and salts thereof, aspartame-acesulfame salt, sucralose, thaumatin, stevia, stevioside, and neohesperidin dihydrochalcone.

73. A process for modulating the taste of a composition comprising adding at least one water-dispersible carotenoid nanoparticle to said composition.

74. The process of claim 73, wherein said at least one water-dispersible carotenoid nanoparticle is used in an amount such that, when said composition is a solid, a concentration of carotenoid or carotenoids of 0.1 to 100 ppm results, and when said composition is a liquid, a concentration of carotenoid or carotenoids of 0.1 to 100 mg/l results.

75. The process of claim 73, wherein said composition is a food, drink, article consumed for pleasure, sweetening agent, animal feed, cosmetic, or pharmaceutical.

76. The process of claim 73, wherein said composition is a low-sugar composition having less than 10 g of sugar per liter or kg.

77. The process of claim 76, wherein said composition is a sugar-free composition.

78. The process of claim 73, wherein said compositions comprises at least one High Intensity Sweetener (HIS) as sweetening agent.

79. The process of claim 73, wherein at least one azo compound comprising at least one azo group and which reduces the bitter taste and aftertaste of said composition is further added to said composition.

80. The process of claim 79, wherein from 0.1 to 100 ppm of said at least one azo compound is added to said composition, based on the total amount of said at least one azo compound and said composition.

81. The process of claim 79, wherein said at least one azo compound is selected from the group consisting of E110, E123, E128, and E129:

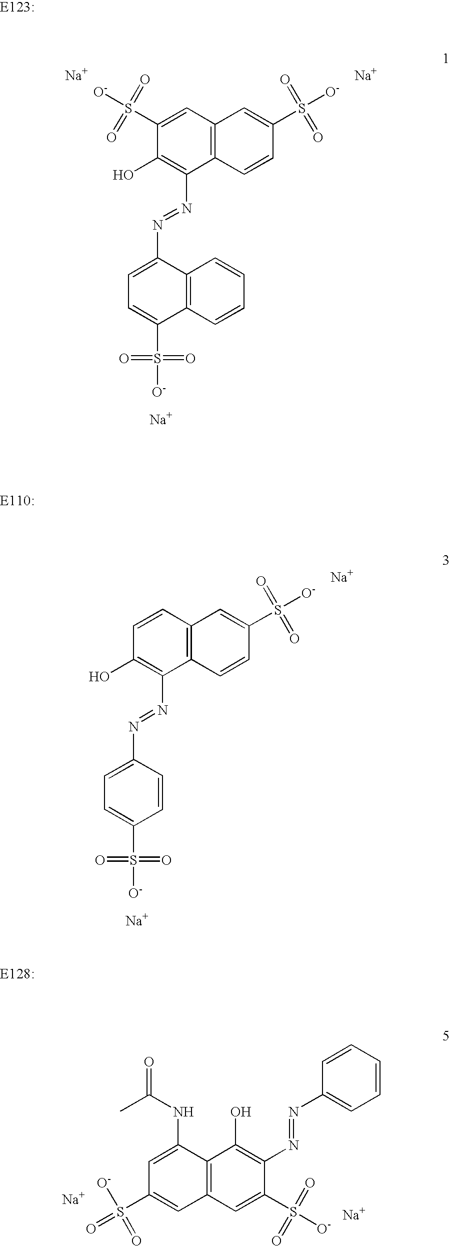

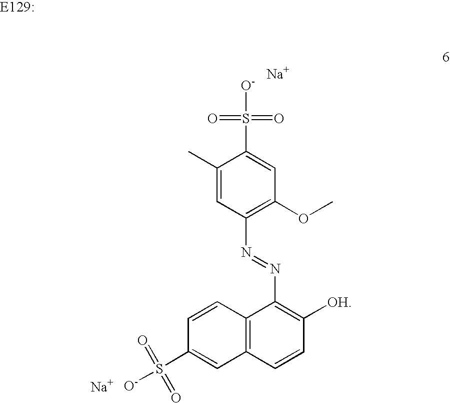

82. The process of claim 78, wherein said at least one HIS is selected from the group consisting of acesulfame-potassium, aspartame, saccharin and salts thereof, cyclamate and salts thereof, aspartame-acesulfame salt, sucralose, thaumatin, and neohesperidin dihydrochalcone.

83. The process of claim 73, wherein the bitter taste and the bitter aftertaste of said composition is reduced as a result of the taste modulation.

84. A taste modulator for a composition which comprises
(A) at least one water-dispersible carotenoid nanoparticle; and
(B) at least one azo compound comprising at least one azo group.

85. The taste modulator of claim 84, wherein the weight ratio of (A) to (B) is in the range of from 10:1 to 1:20.

86. The taste modulator of claim 84, wherein said at least one water-dispersible carotenoid nanoparticle has a particle size of less than 1 μm.

87. The taste modulator of claim 84, wherein said at least one water-dispersible carotenoid nanoparticle is liquid or solid.

88. The taste modulator of claim 87, wherein said at least one water-dispersible carotenoid nanoparticle is solid and the carotenoid or carotenoids in said solid nanoparticle are X-ray amorphous.

89. The taste modulator of claim 87, wherein said at least one water-dispersible carotenoid nanoparticle is liquid and the carotenoid or carotenoids in said liquid nanoparticle are dissolved in a liquid medium or are present in the form of a solubilized mixed micelle.

90. The taste modulator of claim 84, wherein said at least one water-dispersible carotenoid nanoparticle is present in a liquid or solid formulation which comprises at least one additive permitted under food law and/or drugs law.

91. The taste modulator of claim 90, wherein said at least one additive is selected from the group consisting of protective colloids, stabilizers against oxidative breakdown, emulsifiers, oils, plasticizers, compositions against caking, and polyols.

92. The taste modulator of claim 90, said at least one water-dispersible carotenoid nanoparticle is present in a solid formulation which comprises at least one additive permitted under food law and/or drugs law, wherein said at least one water-dispersible carotenoid nanoparticle is embedded in a matrix of said at least one additive.

93. The taste modulator of claim 90, wherein said liquid or solid formulation comprises from 0.5 to 30% by weight of carotenoid or carotenoids, based on the total weight of said liquid or solid formulation.

94. The taste modulator of claim 90, wherein said liquid or solid formulation comprises from 10 to 80% by weight of a protective colloid, based on the total weight of said liquid or solid formulation.

95. The taste modulator of claim 90, wherein said liquid or solid formulation comprises from 20 to 70% by weight of a plasticizer, based on the total weight of said liquid or solid formulation.

96. The taste modulator of claim 84, wherein said at least one azo group is linked to an aryl group.

97. The taste modulator of claim 96, wherein said aryl group is a phenyl group and/or a naphthyl group.

98. The taste modulator of claim 96, wherein said aryl group is substituted by at least one sulfonic acid group and/or at least one hydroxyl group.

99. The taste modulator of claim 96, wherein said at least one azo compound is selected from the group consisting of E110, E123, E128, and E129:

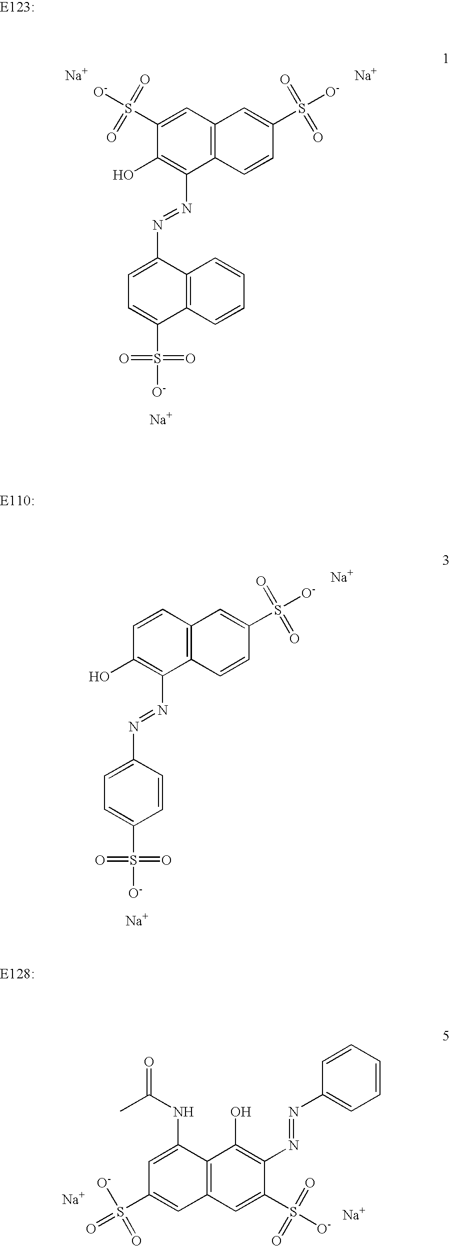

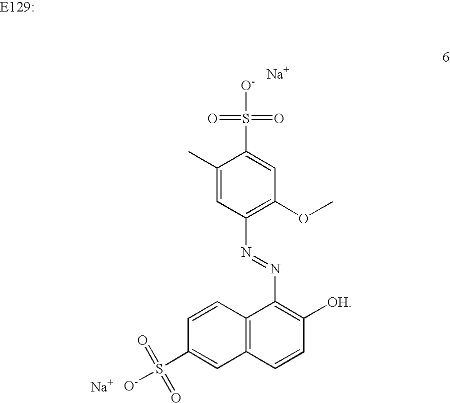

100. The composition of claim 52, wherein said composition is a low-carbohydrate composition.

101. The composition of claim 52, wherein said composition is a carbohydrate-free composition.

102. The composition of claim 52, wherein said composition is an ACK-sweetened drink.

103. The composition of claim 102, wherein said ACK-sweetened drink comprises caffeine.

104. The process of claim 73, wherein said composition is a low-carbohydrate composition.

105. The process of claim 73, wherein said composition is a carbohydrate-free composition.

106. The process of claim 73, wherein said composition is an ACK-sweetened drink.

107. The process of claim 106, wherein said ACK-sweetened drink comprises caffeine.
